(19) United States
(12) Patent Application Publication
(10) Pub. No.: US 2016/0310621 A1
(43) Pub. Date: Oct. 27, 2016

(54) IMAGING PROBES, FORMULATIONS, AND USES THEREOF

(71) Applicants: Charles E. McKenna, Culver City, CA (US); Shuting Sun, Culver City, CA (US); Frank H. Ebetino, Culver City, CA (US); Mark W. Lundy, Culver City, CA (US); Zibo Li, Culver City, CA (US)

(72) Inventors: Charles E. McKenna, Culver City, CA (US); Shuting Sun, Culver City, CA (US); Frank H. Ebetino, Culver City, CA (US); Mark W. Lundy, Culver City, CA (US); Zibo Li, Culver City, CA (US)

(21) Appl. No.: 15/134,112

(22) Filed: Apr. 20, 2016

Related U.S. Application Data
(60) Provisional application No. 62/150,497, filed on Apr. 21, 2015.

(51) Int. Cl. A61K 51/04

(52) U.S. Cl. CPC A61K 51/0489

(57) ABSTRACT

Described herein are single and dual modality bisphosphonate conjugated imaging probes. Also described herein are methods of synthesizing and using the single and dual modality bisphosphonate conjugated imaging probes.

### CROSS-REFERENCE TO RELATED APPLICATIONS

This application claims the benefit of U.S. Provisional Application Ser. No. 62/150,497, filed on Apr. 21, 2015, having the title “Imaging probes for diagnosis of rheumatoid arthritis”, by McKenna et al., the entirety, of which is incorporated herein by reference as if fully set forth herein.

### STATEMENT REGARDING FEDERALLY SPONSORED RESEARCH OR DEVELOPMENT

This invention was made with government support under grant number 1R43AR067021-01A1 awarded by the NIH/NIAMS. The government has certain rights in the invention.

### BACKGROUND

Rheumatoid arthritis (RA) is a progressive, degenerative disease that affects approximately 1% of the population. The disease is auto-immune in nature and causes extensive inflammatory synovial pathology that eventually leads to destructive processes in cartilage, bone, and other associated tissues with ultimate outcomes being disability and joint deformity. It is estimated that the number of people living with RA in the US is over 1 million, with the incidence increasing from 1995 to 2007. Demographic factors such as overall aging of the population will lead to further disease burdens on individuals, as well as on public health in general. In 2010, RA was estimated to cost an additional $19B ($39.2B dollars including intangible costs) in opportunity costs and productivity loss to the US economy.

Currently, there is no cure for RA and although anti-TNF therapies help ease symptoms, reduce inflammation, and slow the progression of the disease, they are fully effective in only ˜60% of patients and are given to affected patients only after other standard therapies have failed. Thus, early diagnosis and constant monitoring of the level of disease activity are key to preventing joint destruction and organ damage. Therefore, an urgent need for improved diagnostic and monitoring procedures and tools for RA and other diseases and disorders that benefit from early diagnosis and continuous monitoring exists.

### SUMMARY

Provided herein are imaging probes that can contain a bisphosphonate and a positron emission tomography (PET) radionuclide, wherein the PET radionuclide is conjugated to the bisphosphonate. The PET radionuclide can be directly conjugated to the bisphosphonate via N-succinimidyl-4-[¹⁸F]fluorobenzoate and an epoxy-containing linker. The PET radionuclide conjugate can be synthesized from N-succinimidyl-4-[¹⁸F]fluorobenzoate bound to a bisphosphonate through an epoxy-containing linker. The PET radionuclide can be selected from the group consisting of: ¹⁸F, ¹¹C, ⁶⁰Cu, ⁶¹Cu, ⁶⁴Cu, ⁸⁶Y, ¹²⁴I, and ⁸⁹Zr. The bisphosphonate can be a nitrogen containing bisphosphonate. The bisphosphonate can be a non-nitrogen containing bisphosphonate. The bisphosphonate can be selected from the group consisting of risedronate, zoledronate, minodronate, pamidronate, neridronate, olpadronate, alendronate, ibandronate and analogues thereof. In some embodiments, an alpha-hydroxyl of the bisphosphonate is substituted with H. In some embodiments, the bisphosphonate can be (2-(pyridin-4-yl)ethane-1,1-diyl)bis(phosphonic acid) or a salt thereof.

The imaging probes provided herein can further contain a fluorescent molecule, wherein the fluorescent molecule can be conjugated to the bisphosphonate via an epoxy-containing linker and wherein the PET radionuclide can be directly conjugated to the fluorescent molecule. The fluorescent molecule can be a boron-dipyrromethene (BPDIPY) dye. The bisphosphonate can be a nitrogen containing bisphosphonate. The bisphosphonate can be a non nitrogen containing bisphosphonate. The bisphosphonate can be selected from the group consisting of risedronate, zoledronate, minodronate, pamidronate, neridronate, olpadronate, alendronate, ibandronate and analogues thereof. In embodiments, an alpha-hydroxyl of the bisphosphonate can be substituted with H. The bisphosphonate can be (2-(pyridin-4-yl)ethane-1,1-diyl)bis(phosphonic acid or a salt thereof. The bisphosphonate can be (1-hydroxy-2-(pyridin-4-yl)ethane-1,1-diyl)bis(phosphonic acid) or a salt thereof.

Also provided herein are methods containing the steps of administering an imaging probe to a subject in need thereof, wherein the imaging probe that can contain a bisphosphonate and a positron emission tomography (PET) radionuclide, wherein the PET radionuclide is conjugated to the bisphosphonate and obtaining an image of at least a portion of the subject using PET scanning. The PET radionuclide can be directly conjugated to the bisphosphonate via N-succinimidyl-4-[¹⁸F]fluorobenzoate and an epoxy-containing linker. The PET radionuclide conjugate can be synthesized from N-succinimidyl-4-[¹⁸F]fluorobenzoate bound to a bisphosphonate through an epoxy-containing linker. The PET radionuclide can be selected from the group consisting of: ¹⁸F, ¹¹C, ⁶⁰Cu, ⁶¹Cu, ⁶⁴Cu, ⁸⁶Y, ¹²⁴I, and ⁸⁹Zr. The bisphosphonate can be a nitrogen containing bisphosphonate. The bisphosphonate can be a non-nitrogen containing bisphosphonate. The bisphosphonate can be selected from the group consisting of risedronate, zoledronate, minodronate, pamidronate, neridronate, olpadronate, alendronate, ibandronate and analogues thereof. In some embodiments, an alpha-hydroxyl of the bisphosphonate is substituted with H. In some embodiments, the bisphosphonate can be (2-(pyridin-4-yl)ethane-1,1-diyl)bis(phosphonic acid) or a salt thereof.

The imaging probes provided herein can further contain a fluorescent molecule, wherein the fluorescent molecule can be conjugated to the bisphosphonate via an epoxy-containing linker and wherein the PET radionuclide can be directly conjugated to the fluorescent molecule. The fluorescent molecule can be a boron-dipyrromethene (BPDIPY) dye. The bisphosphonate can be a nitrogen containing bisphosphonate. The bisphosphonate can be a non nitrogen containing bisphosphonate. The bisphosphonate can be selected from the group consisting of risedronate, zoledronate, minodronate, pamidronate, neridronate, olpadronate, alendronate, ibandronate and analogues thereof. In embodiments, an alpha-hydroxyl of the bisphosphonate can be substituted with H. The bisphosphonate can be (2-(pyridin-4-yl)ethane-1,1-diyl)bis(phosphonic acid or a salt thereof. The bisphosphonate can be (1-hydroxy-2-(pyridin-4-yl)ethane-1,1-diyl)bis(phosphonic acid) or a salt thereof.

The subject in need thereof can have or can be suspected of having, or can be otherwise predisposed to having a bone related disease, wherein the bone related disease can selected from the group of: multiple myeloma, bone metastasis, Paget's disease, steroid induced osteoporosis, osteosarcoma osteoporosis, osteopenia, heterotopic ossification, osteoarthritis, rheumatoid arthritis, a disorder characterized by high bone turnover, and any combination thereof.

### BRIEF DESCRIPTION OF THE DRAWINGS

Further aspects of the present disclosure will be readily appreciated upon review of the detailed description of its various embodiments, described below, when taken in conjunction with the accompanying drawings.

FIGS. 1A and 1B show embodiments of imaging probes.

FIGS. 2A-2C show embodiments of imaging probes having the bisphosphonate risedronate (FIG. 2A), zoledronate (FIG. 2B) and p-PyrEBP (FIG. 2C). Circles in FIG. 2C demonstrate the modified alpha-hydroxy group and the para configuration compared to FIG. 2A. The inset in FIG. 2A shows risedronate, the inset in FIG. 2B shows zoledronate, and the insert in FIG. 2C shows p-PyrEBP.

FIGS. 3A-3F show BODPY-1 (FIG. 3A), BODIPYR6G (FIG. 3B), and BODIPY-3 (FIG. 3C), ¹⁸F BODIPY-1 (FIG. 3D), ¹⁸F BODIPYR6G (FIG. 3E), and ¹⁸F BODIPY-3 (FIG. 3F).

FIGS. 4A-4C show embodiments of BP conjugated imaging probes that lack or have reduced anti-resorptive effects (inactive). FIG. 4A shows an embodiment of an inactive single modality BP conjugated imaging probe. FIGS. 4B, 4C show embodiments of inactive dual modality BP conjugated imaging probes.

FIGS. 5A-5F show embodiments of BP conjugated imaging probes that incorporate risedronate and zoledronate. FIGS. 5A and 5B show embodiments of single modality RIS (FIG. 5A) and ZOL (FIG. 5B) conjugated imaging probes. FIGS. 5C-5F show embodiments of dual modality RIS (FIGS. 5C, 5E) and ZOL (FIGS. 5D, 5F) conjugated imaging probes.

FIGS. 6A-6B show embodiments of a synthesis scheme for producing single and dual modality BP conjugated PET imaging probes.

FIG. 7 shows an embodiment of a synthesis scheme for producing F (cold or hot) labeled BP using N-succinimidyl 4-fluorobenzoate (SFB) coupling.

FIG. 8 shows an embodiment of a synthesis scheme for producing radiolabeled BP via final step radiolabel incorporation.

FIG. 9 shows a graph demonstrating high-performance liquid chromatography (HPLC) radio channel trace of ¹⁸F-RIS-SFB with the standard UV channel chromatogram (inset) for comparison of retention time.

FIGS. 10A-10B show embodiments of two synthesis schemes of a dual modality BP-conjugated imaging probe.

FIG. 11 shows a graph demonstrating ankle thickness in a rat RA model generated by injection of heat killed Mycobacterium tuberculosis (strain H37Ra) suspended in incomplete Freund's adjuvant (HKMT/IFA) treatment (circles) and control rats (squares).

FIGS. 12A-12D show photographic images of control (FIGS. 12A and 12C) and RA model rats (FIGS. 12B and 12D) at day 7 (FIGS. 12A-12B) and at day 16 (FIGS. 12C and 12D) post HKMT/IFA treatment.

FIGS. 13A-13B shows images from a PET scan of a representative control (FIG. 13B) and RA rat (FIG. 13A) taken after i.v. injection of a single modality BP conjugated imaging probe.

FIG. 14 shows a graph demonstrating detection of bone resorption at day 7 and 18 post RA induction.

FIG. 15 shows a graph demonstrating that Na¹⁸F when used as a PET imaging probe does not detect the early bone involvement in RA at day 7.

FIG. 16 shows a PET scan image taken after i.v. injection of a dual modality BP conjugated imaging probe in a normal (non-RA) mouse.

FIG. 17 shows a graph demonstrating joint and major organ radioactivity accumulation quantification from a static PET scan taken at about 1 h post injection injection of a dual modality BP conjugated imaging probe.

FIGS. 18A-18B show images from a fluorescent scan of a representative mouse injected with the BP-conjugated dual modality probe (FIG. 18A) and a mouse injected with the BODIPY dye only (FIG. 18B)

FIG. 19 shows embodiments of synthesis schemes to produce near infrared (NIR) BODIPY dyes.

FIG. 20 shows embodiments of synthesis schemes to produce water-soluble BODIPY dyes.

FIG. 21 shows one embodiment of a synthesis scheme to produce a NIR-BODIPY dual modality BP conjugated imaging probe (for both ¹⁸F and ¹⁹F versions).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, botany and the like, which are within the skill of the art. Such techniques are explained fully in the literature.

Definitions

Unless otherwise specified herein, the following definition are provided.

As used herein, “about,” “approximately,” and the like, when used in connection with a numerical variable, generally refers to the value of the variable and to all values of the variable that are within the experimental error (e.g., within the 95% confidence interval for the mean) or within ±10% of the indicated value, whichever is greater.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term “farm animal” includes a horse, sheep, goat, chicken, pig, cow, donkey, llama, alpaca, turkey, and the like.

As used herein, “control” can refer to an alternative subject or sample used in an experiment for comparison purposes and included to minimize or distinguish the effect of variables other than an independent variable.

As used herein, “radionuclide” refers can be used interchangeably with the terms “radioisotope” and “radioactive isotope” and can refer to an atom that has excess nuclear energy, making it unstable.

As used herein, “analogue,” such as an analogue of a bisphosphonate described herein, can refer to a structurally close member of the parent molecule or an appended parent molecule such as a bisphosphonate.

As used herein, “conjugated” can refer to direct attachment of two or more compounds to one another via one or more covalent or non-covalent bonds. The term “conjugated” as used herein can also refer to indirect attachment of two or more compounds to one another through an intermediate compound, such as a linker.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “pharmaceutically acceptable salt” refers to any acid or base addition salt whose counter-ions are non-toxic to the subject to which they are administered in pharmaceutical doses of the salts.

As used herein, “active agent” or “active ingredient” refers to a component or components of a composition to which the whole or part of the effect of the composition is attributed.

As used herein, “dose,” “unit dose,” or “dosage” refers to physically discrete units suitable for use in a subject, each unit containing a predetermined quantity of an imaging probe composition or formulation calculated to produce the desired response or responses in association with its administration.

As used herein, “derivative” refers to any compound having the same or a similar core structure to the compound but having at least one structural difference, including substituting, deleting, and/or adding one or more atoms or functional groups. The term “derivative” does not mean that the derivative is synthesized from the parent compound either as a starting material or intermediate, although this may be the case. The term “derivative” can include prodrugs, or metabolites of the parent compound. Derivatives include compounds in which free amino groups in the parent compound have been derivatized to form amine hydrochlorides, p-toluene sulfoamides, benzoxycarboamides, t-butyloxycarboamides, thiourethane-type derivatives, trifluoroacetylamides, chloroacetylamides, or formamides. Derivatives include compounds in which carboxyl groups in the parent compound have been derivatized to form methyl and ethyl esters, or other types of esters, amides, hydroxamic acids, or hydrazides. Derivatives include compounds in which hydroxyl groups in the parent compound have been derivatized to form O-acyl, O-carbamoyl, or O-alkyl derivatives. Derivatives include compounds in which a hydrogen bond donating group in the parent compound is replaced with another hydrogen bond donating group such as OH, NH, or SH. Derivatives include replacing a hydrogen bond acceptor group in the parent compound with another hydrogen bond acceptor group such as esters, ethers, ketones, carbonates, tertiary amines, imine, thiones, sulfones, tertiary amides, and sulfides. “Derivatives” also includes extensions of the replacement of the cyclopentane ring, as an example, with saturated or unsaturated cyclohexane or other more complex, e.g., nitrogen-containing rings, and extensions of these rings with various groups.

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, e.g. 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, amino acid, peptide, and polypeptide groups.

As used herein, “suitable substituent” means a chemically and pharmaceutically acceptable group, i.e., a moiety that does not significantly interfere with the preparation of or negate the efficacy of the inventive compounds. Such suitable substituents may be routinely chosen by those skilled in the art. Suitable substituents include but are not limited to the following: a halo, C₁-C₆ alkyl, C₂-C₆ alkenyl, C₁-C₆ haloalkyl, C₁-C₆ alkoxy, C₁-C₆ haloalkoxy, C₂-C₆ alkynyl, C₃-C₈ cycloalkenyl, (C₃-C₈ cycloalkyl)C₁-C₆ alkyl, (C₃-C₈ cycloalkyl)C₂-C₆ alkenyl, (C₃-C₈ cycloalkyl)C₁-C₆ alkoxy, C₃-C₇ heterocycloalkyl, (C₃-C₇ heterocycloalkyl)C₁-C₆ alkyl, (C₃-C₇ heterocycloalkyl) C₂-C₆ alkenyl, (C₃-C₇ heterocycloalkyl)C₁-C₆ alkoxyl, hydroxy, carboxy, oxo, sulfanyl, C₁-C₆ alkylsulfanyl, aryl, heteroaryl, aryloxy, heteroaryloxy, aralkyl, heteroaralkyl, aralkoxy, heteroaralkoxy, nitro, cyano, amino, C₁-C₆ alkylamino, di-(C₁-C₆ alkyl)amino, carbamoyl, (C₁-C₆ alkyl)carbonyl, (C₁-C₆ alkoxy)carbonyl, (C₁-C₆ alkyl)aminocarbonyl, di-(C₁-C₆ alkyl)aminocarbonyl, arylcarbonyl, aryloxycarbonyl, (C₁-C₆ alkyl)sulfonyl, and arylsulfonyl. The groups listed above as suitable substituents are as defined hereinafter except that a suitable substituent may not be further optionally substituted.

The term “alkyl” refers to the radical of saturated aliphatic groups (i.e., an alkane with one hydrogen atom removed), including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl can have 30 or fewer carbon atoms in its backbone (e.g., C₁-C₃₀ for straight chains, and C₃-C₃₀ for branched chains). In other embodiments, a straight chain or branched chain alkyl can contain 20 or fewer, 15 or fewer, or 10 or fewer carbon atoms in its backbone. Likewise, in some embodiments cycloalkyls can have 3-10 carbon atoms in their ring structure. In some of these embodiments, the cycloalkyl can have 5, 6, or 7 carbons in the ring structure.

The term “alkyl” (or “lower alkyl”) as used herein is intended to include both “unsubstituted alkyls” and “substituted alkyls,” the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, phosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons in its backbone structure. Likewise, “lower alkenyl” and “lower alkynyl” have similar chain lengths.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl,” as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In preferred embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy,” as used herein, refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl is an ether or resembles an alkoxyl, such as can be represented by one of —O-alkyl, —O-alkenyl, and —O-alkynyl. The terms “aroxy” and “aryloxy”, as used interchangeably herein, can be represented by —O-aryl or O-heteroaryl, wherein aryl and heteroaryl are as defined below. The alkoxy and aroxy groups can be substituted as described above for alkyl.

The terms “amine” and “amino” (and its protonated form) are art-recognized and refer to both unsubstituted and substituted amines, e.g., a moiety that can be represented by the general formula:

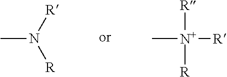

wherein R, R′, and R″ each independently represent a hydrogen, an alkyl, an alkenyl, —(CH2)ₘ—R_{C} or R and R′ taken together with the N atom to which they are attached complete a heterocycle having from 4 to 8 atoms in the ring structure; R_{C} represents an aryl, a cycloalkyl, a cycloalkenyl, a heterocycle or a polycycle; and m is zero or an integer in the range of 1 to 8. In some embodiments, only one of R or R′ can be a carbonyl, e.g., R, R′ and the nitrogen together do not form an imide. In other embodiments, the term “amine” does not encompass amides, e.g., wherein one of R and R′ represents a carbonyl. In further embodiments, R and R′ (and optionally R″) each independently represent a hydrogen, an alkyl or cycloakly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R and R′ is an alkyl group.

The term “amido” is art-recognized as an amino-substituted carbonyl and includes a moiety that can be represented by the general formula:

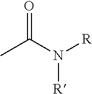

wherein R and R′ are as defined above.

As used herein, “Aryl” refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine, pyrimidine, and the like. Those aryl groups having heteroatoms in the ring structure may also be referred to as “aryl heterocycles” or “heteroaromatics.” The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl. One or more of the rings can be substituted as defined above for “aryl.”

The term “aralkyl,” as used herein, refers to an alkyl group substituted with an aryl group (e.g., an aromatic or heteroaromatic group).

The term “aralkyloxy” can be represented by —O-aralkyl, wherein aralkyl is as defined above.

The term “carbocycle,” as used herein, refers to an aromatic or non-aromatic ring(s) in which each atom of the ring(s) is carbon.

“Heterocycle” or “heterocyclic,” as used herein, refers to a monocyclic or bicyclic structure containing 3-10 ring atoms, and in some embodiments, containing from 5-6 ring atoms, wherein the ring atoms are carbon and one to four heteroatoms each selected from the following group of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like. The terms “heterocycle” or “heterocyclic” can be used to describe a compound that can include a heterocyle or heterocyclic ring.

The term “carbonyl” is art-recognized and includes such moieties as can be represented by the general formula:

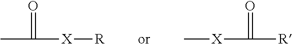

wherein X is a bond or represents an oxygen or a sulfur, and R and R′ are as defined above. Where X is an oxygen and R or R′ is not hydrogen, the formula represents an “ester”. Where X is an oxygen and R is as defined above, the moiety is referred to herein as a carboxyl group, and particularly when R is a hydrogen, the formula represents a “carboxylic acid.” Where X is an oxygen and R′ is hydrogen, the formula represents a “formate.” In general, where the oxygen atom of the above formula is replaced by sulfur, the formula represents a “thiocarbonyl” group. Where X is a sulfur and R or R′ is not hydrogen, the formula represents a “thioester.” Where X is a sulfur and R is hydrogen, the formula represents a “thiocarboxylic acid.” Where X is a sulfur and R′ is hydrogen, the formula represents a “thioformate.” On the other hand, where X is a bond, and R is not hydrogen, the above formula represents a “ketone” group. Where X is a bond, and R is hydrogen, the above formula represents an “aldehyde” group.

The term “heteroatom” as used herein means an atom of any element other than carbon or hydrogen. Exemplary heteroatoms include, but are not limited to, boron, nitrogen, oxygen, phosphorus, sulfur, silicon, arsenic, and selenium. Heteroatoms, such as nitrogen, can have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

As used herein, the term “nitro” refers to —NO_{2;} the term “halogen” designates —F, —Cl, —Br, or —I; the term “sulfhydryl” refers to —SH; the term “hydroxyl” refers to —OH; and the term “sulfonyl” refers to —SO₂—.

As used herein, “effective amount” refers to the amount of a composition described herein or pharmaceutical formulation described herein that will elicit a desired biological or medical response of a tissue, system, animal, plant, protozoan, bacteria, yeast or human that is being sought by the researcher, veterinarian, medical doctor or other clinician. The desired biological response can be modulation of bone formation and/or remodeling, including but not limited to modulation of bone resorption and/or uptake of the imaging probes described herein. The effective amount will vary depending on the exact chemical structure of the composition or pharmaceutical formulation, the causative agent and/or severity of the infection, disease, disorder, syndrome, or symptom thereof being treated or prevented, the route of administration, the time of administration, the rate of excretion, the drug combination, the judgment of the treating physician, the dosage form, and the age, weight, general health, sex and/or diet of the subject to be treated. As used there in the effective amount can refer to the amount of the probe described herein that can be effective as a P.E.T. imaging probe, a bone specific P.E.T. imaging probe, and/or effective to diagnose and/or treat one or more symptoms of multiple myeloma, bone metastasis, osteosarcoma, arthritis (including but not limited to rheumatoid arthritis and osteoarthritis) Paget's disease, steroid induced osteoporosis, osteoporosis, osteopenia, heterotopic ossification, and/or another disorder characterized by high bone turnover in a subject in need thereof.

As used herein, “therapeutic” generally can refer to treating, healing, and/or ameliorating a disease, disorder, condition, or side effect, or to decreasing in the rate of advancement of a disease, disorder, condition, or side effect. The term also includes within its scope enhancing normal physiological function, palliative treatment, and partial remediation of a disease, disorder, condition, side effect, or symptom thereof.

As used herein, the terms “treating” and “treatment” can refer generally to obtaining a desired pharmacological and/or physiological effect. The effect may be prophylactic in terms of preventing or partially preventing a disease, symptom or condition thereof.

As used herein, “synergistic effect,” “synergism,” or “synergy” refers to an effect arising between two or more molecules, compounds, substances, factors, or compositions that is greater than or different from the sum of their individual effects.

As used herein, “additive effect” refers to an effect arising between two or more molecules, compounds, substances, factors, or compositions that is equal to or the same as the sum of their individual effects.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

Discussion

Rheumatoid arthritis (RA) is a major form of inflammatory arthritis that affects people of all ages, races and genders. It is estimated that the number of people living with RA in the US is over 1 million, with the incidence increasing from 1995 to 2007. Demographic factors such as overall aging of the population will lead to further disease burdens on individuals, as well as on public health in general. In 2010, RA was estimated to cost an additional $19B ($39.2B dollars including intangible costs) in opportunity costs and productivity loss to the US economy. RA is an autoimmune disease and characterized by an abnormal immune responses that damage the cartilage and bone in joints throughout the body and cause inflammation in many other organs.

RA is currently regarded as an incurable disease. Indeed, even the notion that pharmacotherapy was actually effective in delaying or modifying the disease course was debated until late in the last century. The use of glucocorticoids, anti-inflammatory agents, and other therapies was rightly viewed as a method of controlling or minimizing symptoms without large effects on the progression of the disease. In the 1990s, advances in disease assessment, combined with use of new agents, such as methotrexate began a radical change in the treatment strategy for RA. This accelerated with the clinical trial of the first biological agent for RA treatment targeting tumor necrosis factor in 1994, and currently there are at least nine biologic drugs targeting various components of the underlying pathology of the disease. These agents have improved outcomes for patients with RA and the goal of therapy is now remission of disease. Interestingly, although biologics target several key disease cascades, none achieves remission in more than approximately 50-60% of patients. Because of this relatively low response rate to individual drugs and the irreversibility of the joint damage caused by the disease, the current paradigm is to frequently assess the disease state to switch therapeutic modality, especially in early disease.

With the current focus on achieving remission, it has become essential to monitor the state of disease strictly. The workhorse of RA monitoring has been physician assessment of joints assisted by classic radiographical (x-ray) imaging. As disease-modifying anti-rheumatic drug (DMARD) therapy has brought patients into states of remission, a greater emphasis is being placed on finding novel imaging modalities that can detect disease in sub-clinical states. Conventional radiography cannot easily detect the earliest disease stages where inflammation is occurring in the soft tissue of the joint and poorly predicts disease progression in these patients. Magnetic resonance imaging (MRI) and ultrasound imaging both have shown the ability to detect in both soft tissue changes and bone erosion and are more sensitive than radiography for monitoring disease progression and in diagnosis and treatment outcome assessment. Other imaging methods such as positron emission tomography (PET), scintigraphy and digital X-ray radiogammetry (DXA) have been used in only a limited number of trials with the goal of predicting treatment response and outcomes. There is thus a large clinical need for an imaging method that will reliably show disease activity early in RA for both diagnosing and treatment monitoring.

With the aforementioned deficiencies of the current imaging techniques in mind, described herein are imaging probes that can have a bisphosphonate directly or indirectly conjugated to a PET radionuclide. The imaging probes described herein can be useful for PET scanning of subjects having RA, other bone diseases or disorders, and/or subjects having multiple myeloma. The imaging probes described herein can further be used to diagnose and monitor treatment regimens in subjects in need thereof. The imaging probes described herein can have the advantage of being targeted to both forming and non-forming bone surfaces, which can include resorption surfaces. In particular, whereas current imaging probes such as sodium fluoride target sites where bone formation occurs. ¹⁸F containing bisphosphonates have the unique ability to attach to bone resorption sites in addition to formation sites. This can be important in diseases driven primarily by resorptive activity on bone such as multiple myeloma, bone metastases, osteosarcoma, and early onset arthritis. Furthermore, the particular components of the imaging probes described herein can facilitate end-reaction or late-reaction radiolabeling of bisphosphonates and thus can have the advantage of incorporating radionuclides with shorter half-lives, such as ¹⁸F. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Bisphosphonate Conjugate Imaging Probes and Formulations Thereof

Conventional methods for RA diagnosis and treatment include several structural imaging techniques, notably, plain radiography, bone scintigraphy magnetic resonance imaging, and ultrasound. Improvements in the spatial resolution of these techniques have made it possible to detect bone erosions within about 6-8 weeks of onset of RA symptoms. However, anatomic imaging does not reveal the underlying biomolecular abnormalities in RA. Thus, the earliest changes that precede bone and cartilage destruction cannot be imaged using these conventional techniques.

PET imaging shows promise for use in the diagnosis and treatment of RA and other diseases. Sodium ¹⁸F (Na¹⁸F) probes have been developed; however, they are specific to bone formation sites. As such, Na¹⁸F do not permit imaging of bone resorption sites, which are important for diagnosing and treating bone resorptive disorders.

Bisphosphonates, including the modern generation of nitrogen-containing bisphosphonates, such as risedronate (RIS) and zoledronate (ZOL) have an affinity for bone are used for treating of resorptive diseases such as osteoporosis, Paget's disease, osteolytic lesions in multiple myeloma, and bone metastases from solid tumors. More recently bisphosphonates have been identified as a potential additional treatment for RA but due to a lack of imaging techniques, the concentration and distribution of bisphosphonates in joints and associated structures in patients with RA is unknown.

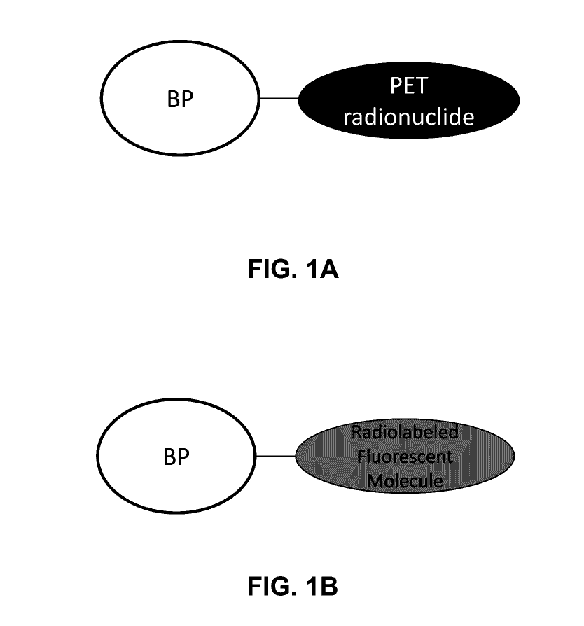

Described herein are imaging probes having a bisphosphonate (BP) conjugated, either directly or indirectly, to a radionuclide (FIGS. 1A and 1B). In some embodiments, the imaging probe can contain a fluorescent molecule that can be conjugated to the bisphosphonate (FIG. 1B). Also described herein are formulations of the imaging probes and methods of making the imaging probes.

Bisphosphonate Conjugate Imaging Probes

The imaging probe can contain a BP. The BP can be a nitrogen containing BP. Suitable bisphosphonates include, but are not limited to, risedronate, zoledronate, minodronate, pamidronate, neridronate, olpadronate, alendronate, ibandronate, and analogues thereof. Additional suitable bisphosphonates include, but are not limited to non nitrogen-containing analogue such as etidronate, medronate, hydroxymethylenediphosphonate (MHDP), tiludronate, and clodronate. The imaging probe can further contain a radionuclide conjugated, either directly or indirectly (such as through a fluorescent molecule), to the BP (e.g. FIGS. 1A-1B). The radionuclide can be suitable for PET imaging. Suitable radionuclides include, but are not limited to, ¹⁸F, ¹¹C, ⁶⁰Cu, ⁶¹Cu, ⁶⁴Cu, ⁸⁶Y, ¹²⁴I, ⁸⁹Zr. The imaging probes described herein can have a radioactivity ranging from 5 mCi to 15 mCi. The radionuclide can be conjugated to the BP via a covalent linker. Suitable linkers include, but are not limited to an epichlorohydrin, oxiran-2-ylmethanamine, tert-butyl (oxiran-2-ylmethyl)carbamate, epoxy-containing linkers, azido-containing linkers, alkyne-containing linkers. In some embodiments, the radionuclide-bearing molecule can be N-succinimidyl-4-fluorobenzoate (SFB), 4-fluorobenzamido-N-ethylamino-maleimide (FBEM), Fluoro-5-methyl-1-B-D-arabinofuranosyluracil (FMAU), and a compound described in U.S. Pat. No. 8,912,319, which is incorporated by reference herein as if expressed in its entirety.

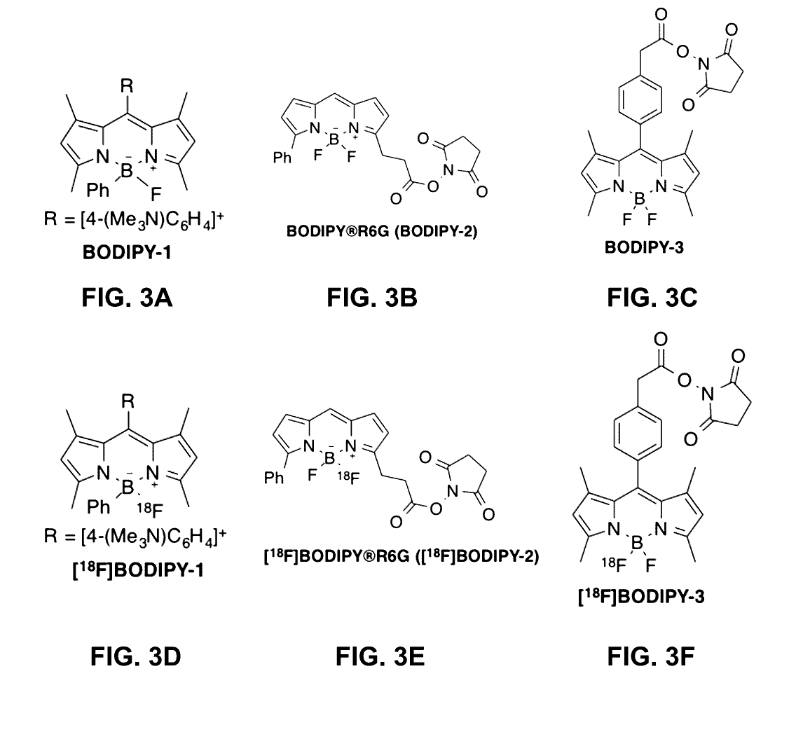

The imaging probe can further contain a radiolabeled fluorescent molecule conjugated to the BP. The radiolabeled fluorescent molecule can be conjugated to the BP via a linker. Suitable linkers include, but are not limited to, epoxy-containing linkers (e.g., a tert-butyl (oxiran-2-ylmethyl)carbamate and any others described in in U.S. Pat. No. 8,431,714), azido-containing linkers, alkyne-containing linkers. In some of the embodiments containing a radiolabeled fluorescent molecule, the BP is not also directly conjugated to a radionuclide via a linker as described above. Suitable fluorescent molecules include, but are not limited to BODIPY (e.g., FIGS. 3A, 3B, 3C). In embodiments, the radiolabeled fluorescent molecule can contain a radionuclide. The radionuclide can be suitable for PET imaging. Suitable radionuclides include, but are not limited to, ¹⁸F, ¹¹C, ⁶⁰Cu, ⁶¹Cu, ⁶⁴Cu, ⁸⁶Y, ¹²⁴I, ⁸⁹Zr. In some embodiments, the radiolabeled fluorescent molecule can be [¹⁸F]BODIPY (e.g., FIGS. 3D, 3E, 3F) or other compound described in U.S. Pat. App. Pub. No.: 2015/0297760.

The anti-resorptive effect of nitrogen-containing BPs, such as RIS and ZOL, can be attributed to the structure of their nitrogen-containing substituent. The bone affinity of BPs can be attributed mainly to the two phosphate groups of BPs. Thus, the anti-resorptive effect of nitrogen-containing BPs is distinct from their bone affinity. The imaging probes described herein can be tuned to have an anti-resorptive effect, to not have an anti-resorptive effect, or to have a partial anti-resorptive effect. It will be understood that the partial anti-resorptive effect refers to any anti-resorptive effect between no effect and the effect equivalent to the unmodified BP.

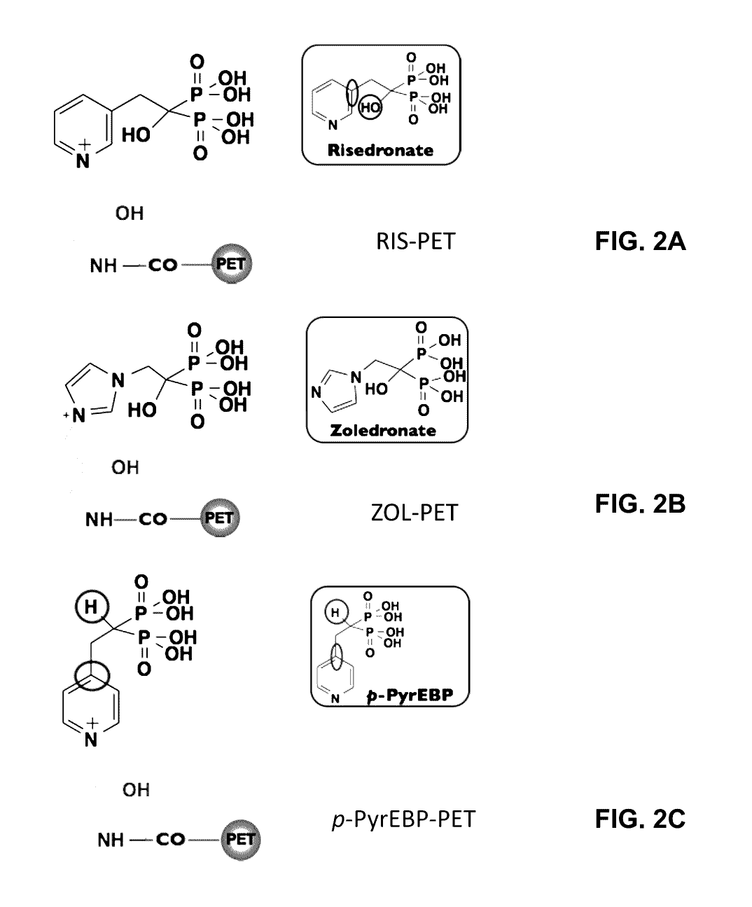

The BP can have an alpha-hydroxy group (e.g., FIGS. 2A, 2B, RIS or ZOL). In some embodiments, the BP can be modified by substituting or removing the alpha-hydroxy group (FIG. 2C, p-PyrEBP). Removal or substitution of the alpha-hydroxyl group can reduce or eliminate the anti-resorptive effect of the BP as compared to an unmodified equivalent BP. As such, in some embodiments, the imaging probe can contain a BP that lacks the alpha-hydroxy group or has a substituted alpha-hydroxy group. Suitable substitutions include, but are not limited to, H, alkyl, aryl, alkyl aryl. Further, the additional molecules that are conjugated to BP can affect the anti-resorptive effect. For example, when the radionuclide, radiolabeled fluorescent molecule, and/or linker is conjugated to a BP having a para-substituted side chain, the anti-resorptive effect can be significantly reduced or eliminated (FIG. 2C, p-PyrEBP-PET). Finally, at the concentration used for PET imaging, all BP-PET imaging probes can be considered without anti-resorptive effect (FIG. 2, RIS-PET, ZOL-PET, and p-PyrEBP-PET). In some embodiments, the BP can be modified to include both an alpha hydroxyl deletion or substitution and a para-substituted side chain.

Methods of Making the Bisphosphonate Conjugate Imaging Probes

In addition to techniques generally known to those of skill in the art, the following methods can be employed to synthesize the imaging probes. Generally, it is desired to incorporate the radionuclide at a step at or near the end of synthesis to avoid loss of radioactivity prior to use. In brief, the BP can be conjugated to a radionuclide or radiolabeled fluorescent molecule via reacting the BP with a radiolabeled linking element or other compound via N-succinimidyl ester coupling. For example, the imaging probes described herein can be made by methods that can include one or more techniques described in Li et al. 2011. Chem. Comm. 47: 9324-9326, which is incorporated by reference herein as if expressed in its entirety. Other methods and techniques that can be used to synthesize the compounds described herein will be appreciated in view of the Examples provided below.

Formulations of the Bisphosphonate Conjugate Imaging Probes

Also described herein are formulations, including pharmaceutical formulations, which can contain an amount of an imaging probe described elsewhere herein. The amount can be an effective amount. Formulations, including pharmaceutical formulations can be formulated for delivery via a variety of routes and can contain a pharmaceutically acceptable carrier. Techniques and formulations generally can be found in Remmington's Pharmaceutical Sciences, Meade Publishing Co., Easton, Pa. (20^{th} Ed., 2000), the entire disclosure of which is herein incorporated by reference. For systemic administration, an injection is useful, including intramuscular, intravenous, intraperitoneal, and subcutaneous. For injection, the therapeutic compositions of the invention can be formulated in liquid solutions, for example in physiologically compatible buffers such as Hank's solution or Ringer's solution. In addition, the imaging probes and/or components thereof can be formulated in solid form and redissolved or suspended immediately prior to use. Lyophilized forms are also included. Formulations, including pharmaceutical formulations, of the imaging probes can be characterized as being at least sterile and pyrogen-free. These formulations include formulations for human and veterinary use.

Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxyl methylcellulose, and polyvinyl pyrrolidone, which do not deleteriously react with the imaging probe.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the imaging probe.

A pharmaceutical formulation can be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Formulations, including pharmaceutical formulations, suitable for injectable use can include sterile aqueous solutions (where water soluble) or dispersions and sterile powders for the extemporaneous preparation of sterile injectable solutions or dispersions. For intravenous administration, suitable carriers can include physiological saline, bacteriostatic water, Cremophor EM™ (BASF, Parsippany, N.J.) or phosphate buffered saline (PBS). Injectable pharmaceutical formulations can be sterile and can be fluid to the extent that easy syringability exists. Injectable pharmaceutical formulations can be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyetheylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it can be useful to include isotonic agents, for example, sugars, polyalcohols such as mannitol, sorbitol, sodium chloride in the composition.

Sterile injectable solutions can be prepared by incorporating any of the imaging probes described herein in an amount in an appropriate solvent with one or a combination of ingredients enumerated herein, as required, followed by filtered sterilization. Generally, dispersions can be prepared by incorporating the imaging probe into a sterile vehicle which contains a basic dispersion medium and the required other ingredients from those enumerated herein. In the case of sterile powders for the preparation of sterile injectable solutions, examples of useful preparation methods are vacuum drying and freeze-drying which yields a powder of the active ingredient plus any additional desired ingredient from a previously sterile-filtered solution thereof.

Systemic administration can also be by transmucosal or transdermal means. For transmucosal or transdermal administration, penetrants appropriate to the barrier to be permeated can be used in the formulation. Such penetrants are generally known in the art, and include, for example, for transmucosal administration, detergents, bile salts, and fluidic acid derivatives. Transmucosal administration can be accomplished through the use of nasal sprays or suppositories. For transdermal administration, the imaging probes can be formulated into ointments, salves, gels, or creams as generally known in the art. In some embodiments, the imaging probes can be applied via transdermal delivery systems, which can slowly release the imaging probe for percutaneous absorption. Permeation enhancers can be used to facilitate transdermal penetration of the active factors in the conditioned media. Transdermal patches are described in for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

For oral administration, a formulation as described herein can be presented as capsules, tablets, powders, granules, or as a suspension or solution. The formulation can contain conventional additives, such as lactose, mannitol, cornstarch or potato starch, binders, crystalline cellulose, cellulose derivatives, acacia, cornstarch, gelatins, disintegrators, potato starch, sodium carboxymethylcellulose, dibasic calcium phosphate, anhydrous or sodium starch glycolate, lubricants, and/or or magnesium stearate.

For parenteral administration (i.e., administration through a route other than the alimentary canal), the formulations described herein can be combined with a sterile aqueous solution that is isotonic with the blood of the subject. Such a formulation can be prepared by dissolving the active ingredient (e.g. the imaging probe) in water containing physiologically-compatible substances, such as sodium chloride, glycine and the like, and having a buffered pH compatible with physiological conditions, so as to produce an aqueous solution, then rendering the solution sterile. The formulation can be presented in unit or multi-dose containers, such as sealed ampoules or vials. The formulation can be delivered by injection, infusion, or other means known in the art.

For transdermal administration, the formulation described herein can be combined with skin penetration enhancers, such as propylene glycol, polyethylene glycol, isopropanol, ethanol, oleic acid, N-methylpyrrolidone and the like, which increase the permeability of the skin to the nucleic acid vectors of the invention and permit the nucleic acid vectors to penetrate through the skin and into the bloodstream. The formulations and/or compositions described herein can be further combined with a polymeric substance, such as ethylcellulose, hydroxypropyl cellulose, ethylene/vinyl acetate, polyvinyl pyrrolidone, and the like, to provide the composition in gel form, which can be dissolved in a solvent, such as methylene chloride, evaporated to the desired viscosity and then applied to backing material to provide a patch.

Dosage Forms

The imaging probes and formulations thereof described herein can be provided in unit dose form such as a tablet, capsule, or single-dose injection or infusion vial. Where appropriate, the dosage forms described herein can be microencapsulated. The dosage form can also be prepared to prolong or sustain the release of any ingredient. In some embodiments, the complexed active agent can be the ingredient whose release is delayed. In other embodiments, the release of an auxiliary ingredient is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, Pa.: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Coatings may be formed with a different ratio of water soluble polymer, water insoluble polymers, and/or pH dependent polymers, with or without water insoluble/water soluble non polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Effective Amounts

The formulations can contain an effective amount of an imaging probe (effective for generating an image via PET scanning) described herein. In some embodiments, the effective amount ranges from about 0.001 pg to about 1,000 g or more of the imaging probe described herein. In some embodiments, the effective amount of the imaging probe described herein can range from about 0.001 mg/kg body weight to about 1,000 mg/kg body weight. In yet other embodiments, the effective amount of the imaging probe can range from about 1% w/w to about 99% or more w/w, w/v, or v/v of the total formulation. The effective amount of the imaging probe can range from 0.0125-0.6 nmol (e.g. for but not limited to mice), 0.125-4 nmol (e.g. for but not limited to rats), 1.25-40 nmol (e.g. for but not limited to human). The effective amount of radioactivity of the imaging probe (i.e. the amount of radioactivity in the dose delivered to the subject effective to generate a PET image) can range from 50 μCi to 300 μCi (e.g. for but not limited to mice), 500 μCi-2 mCi (e.g. for but not limited to rats), and 5-20 mCi (for but not limited to human). In embodiments, the specific radioactivity can range from 500 mCi/μmol-4 Ci/μmol. In some embodiments the effective amount of radioactivity of the imaging probe can range from about 300 μCi to about 20 mCi or more. In some embodiments, the effective amount of radioactivity of the imaging probe ranges from about 500 μCi to about 2 mCi. In some embodiments, the effective amount of radioactivity of the imaging probe can range from about 8 to about 12 mCi.

Methods of Using the Bisphosphonate Conjugate Imaging Probes

An amount, including an effective amount, of the imaging probes and formulations thereof described herein can be administered to a subject in need thereof. In some embodiments the subject in need thereof can have a disease, disorder, or a symptom thereof. In some embodiments, the subject in need thereof can be suspected of having or is otherwise predisposed to having a disease, disorder, or a symptom thereof. In embodiments, the disease or disorder can be a bone and/or a joint disease or disorder, including but not limited to RA, multiple myeloma, bone metastases, cancers metastatic to bone, osteosarcomas, Paget's disease, steroid induced osteoporosis, osteoporosis, osteopenia, heterotopic ossification, osteoarthritis, rheumatoid arthritis, arthritis, and/or other diseases of high bone turnover. After administration to a subject in need thereof, the subject can undergo a suitable imaging technique, such as PET scanning, on one or more regions of the body. One skilled in the art will appreciate the techniques applied in PET scanning.

Administration of the imaging probes is not restricted to a single route, but can encompass administration by multiple routes. For instance, exemplary administrations by multiple routes include, among others, a combination of intradermal and intramuscular administration, or intradermal and subcutaneous administration. Multiple administrations can be sequential or concurrent. Other modes of application by multiple routes will be apparent to the skilled artisan.

The pharmaceutical formulations can be administered to a subject by any suitable method that allows the agent to exert its effect on the subject in vivo. For example, the formulations and other compositions described herein can be administered to the subject by known procedures including, but not limited to, by oral administration, sublingual or buccal administration, parenteral administration, transdermal administration, via inhalation, via nasal delivery, vaginally, rectally, and intramuscularly. The formulations or other compositions described herein can be administered parenterally, by epifascial, intracapsular, intracutaneous, subcutaneous, intradermal, intrathecal, intramuscular, intraperitoneal, intrasternal, intravascular, intravenous, parenchymatous, and/or sublingual delivery. Delivery can be by injection, infusion, catheter delivery, or some other means, such as by tablet or spray.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Synthesis of BP Conjugated Imaging Probes

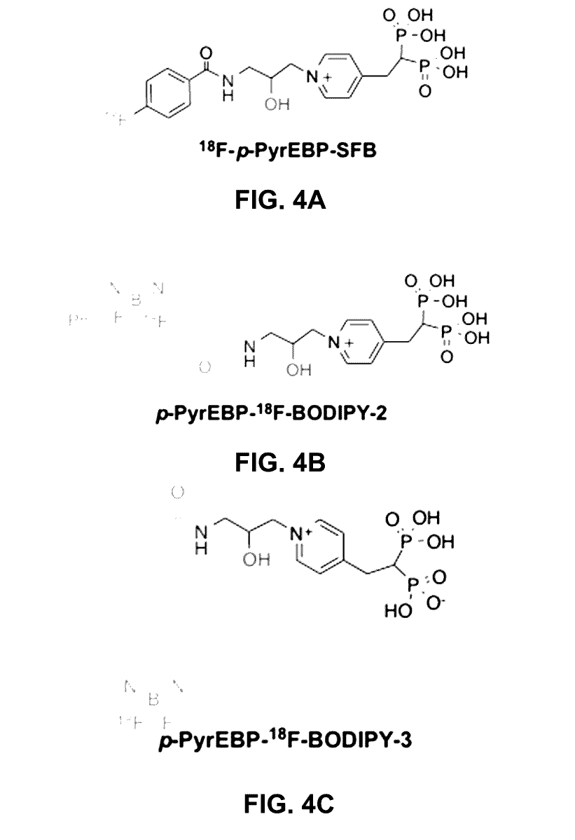

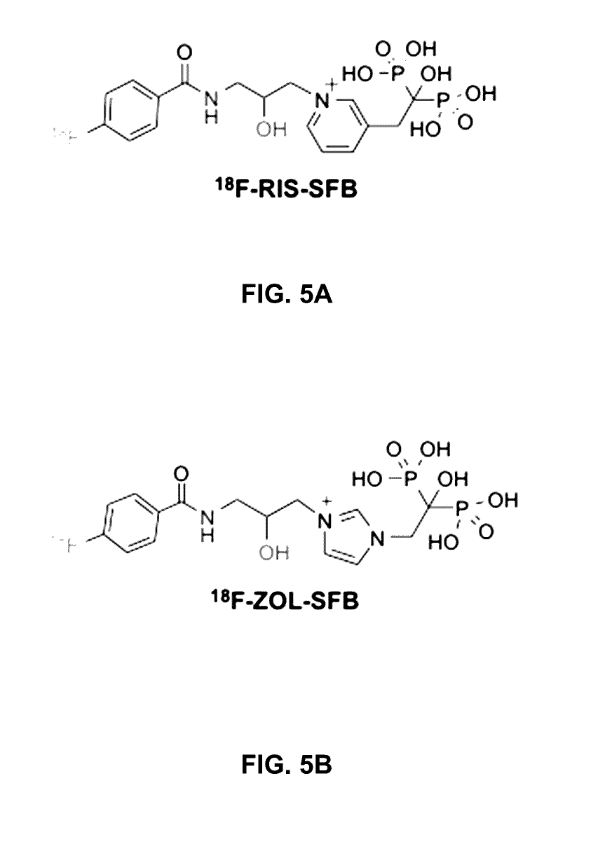

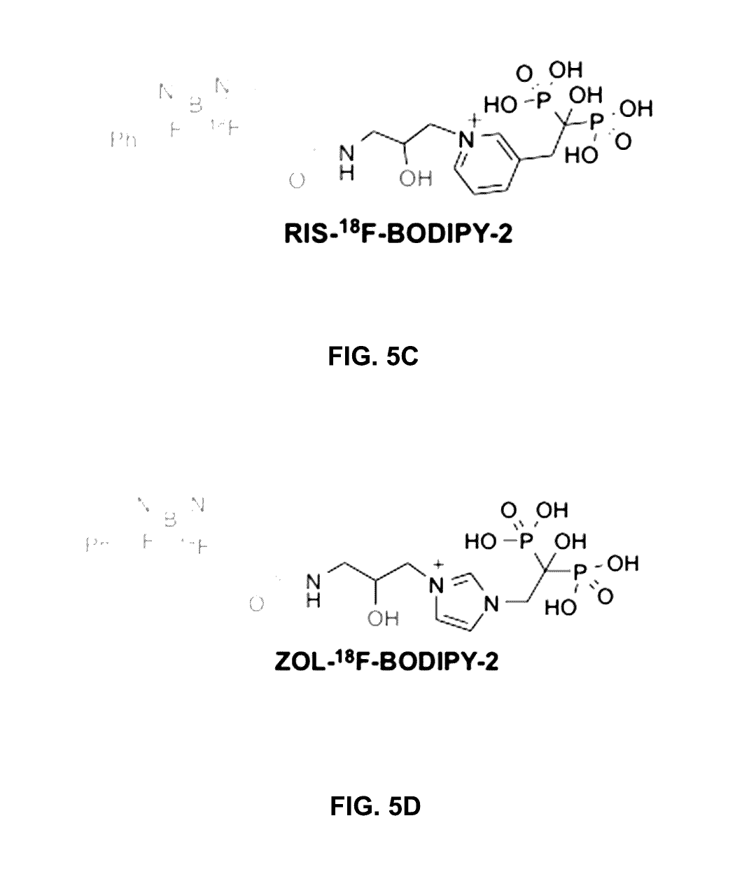

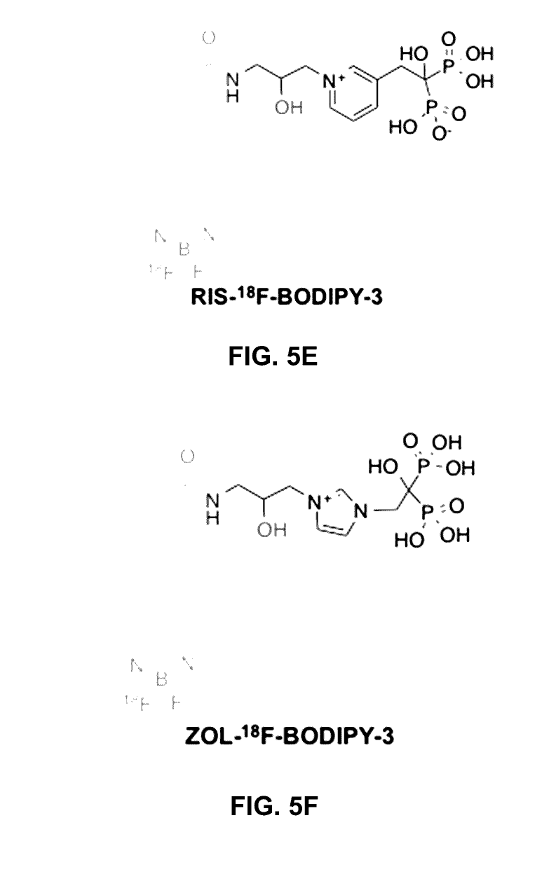

Radiolabeled single and dual modality BP conjugated imaging probes were synthesized. FIGS. 4A-4C show the formulas of the synthesized biologically inactive single modality BP conjugated imaging probe (FIG. 4A) and inactive dual modality BP conjugated imaging probes (FIGS. 4B, 4C). FIGS. 5A-5F show the formulas of single modality imaging probes (FIGS. 5A-5B) and dual modality probes (FIGS. 5C-5F) incorporating RIS (FIGS. 5A, 5C, 5E) and ZOL (FIGS. 5B, 5D, 5F).

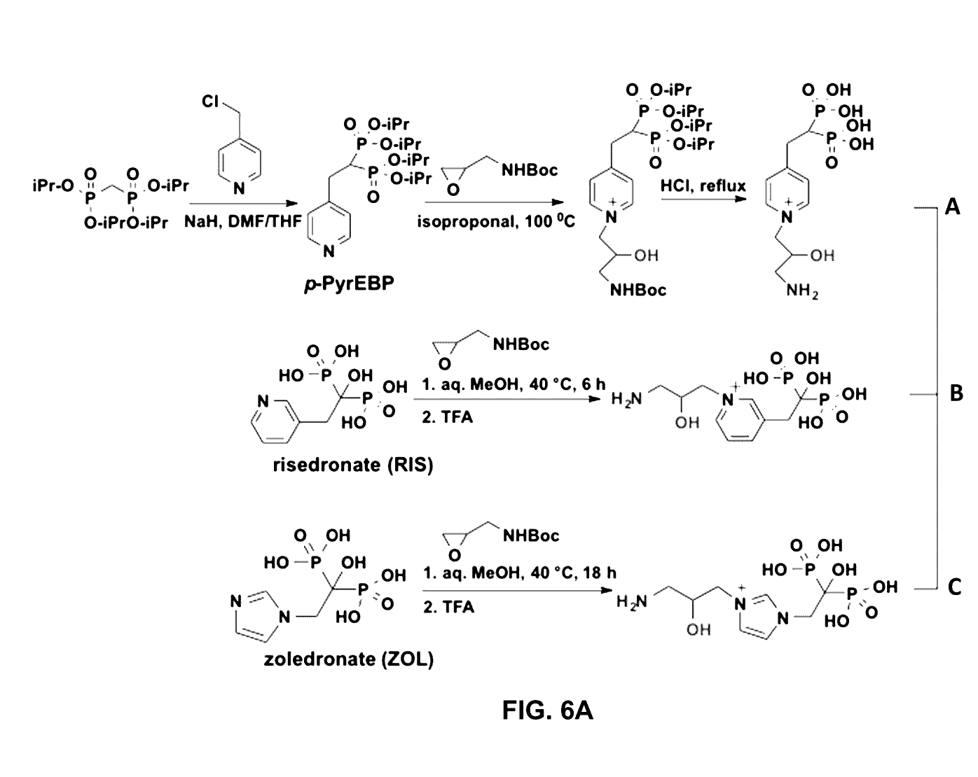

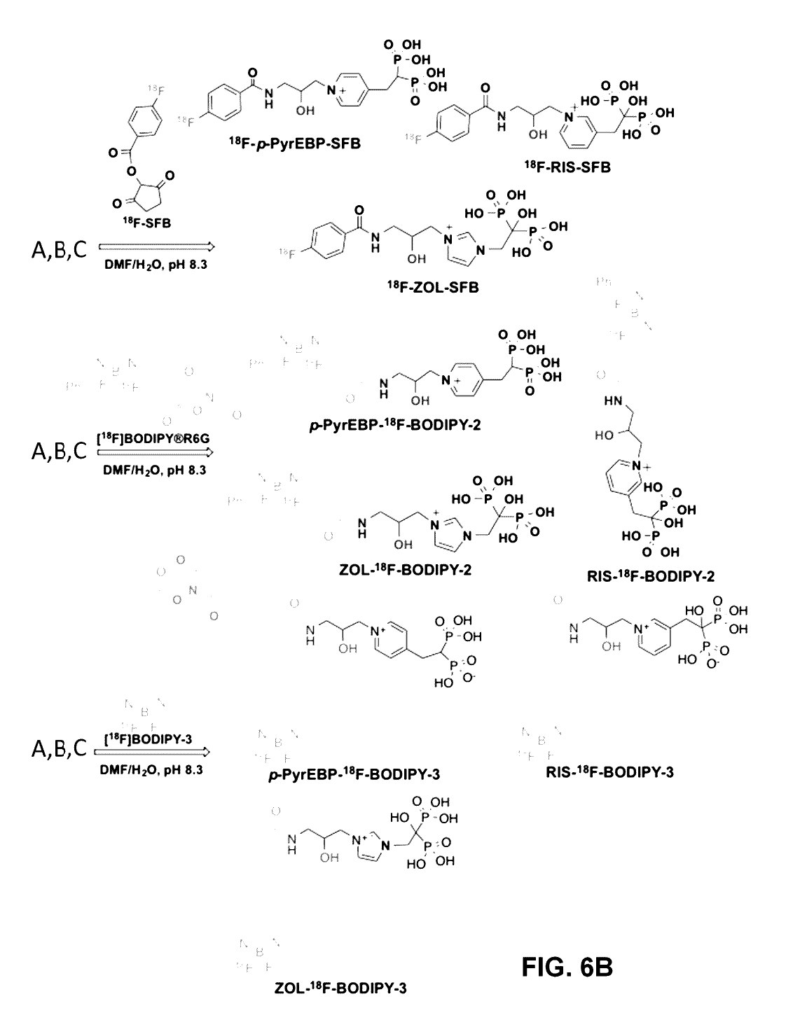

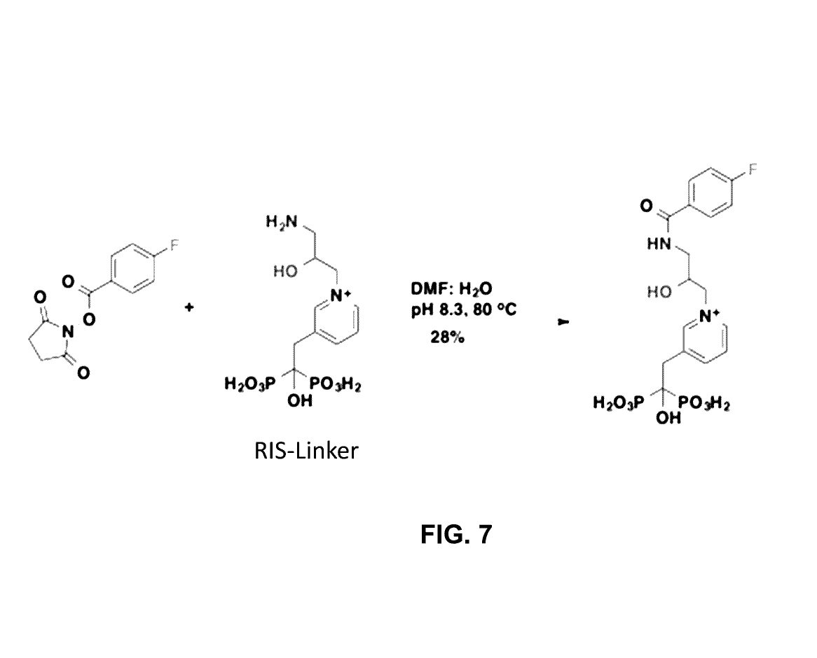

The compounds were synthesized using an N-succinimidyl ester mediate coupling. The synthesis schemes are shown in FIGS. 6A-6B. Generally, the single modality imaging probes were using 3 steps: (1) generating ¹⁸F; (2) incorporating the ¹⁸F label into the SFB (Vaidyanathan, G. and M. R. Zalutsky. Synthesis of N-succinimidyl 4{18F]fluorobenzoate, an agent for labeling proteins and peptides with 18F. Nat. Protoc. 2006. 1(4):1655-1661. (3) N-succinimidyl ester coupling of a BP to the radiolabeled SFB. FIG. 7 demonstrates the approach using cold F; however, the same approach applies to hot F (¹⁸F) with the additional step of generating the radioactive F. Using this approach, radiolabeled SFB was initially synthesized with a radioactivity of about 1-1.5 Cu using an automated GE FxFN module. The conjugation of the radiolabeled SFB to the BP proceeded with a greater than about 80% labeling yield.

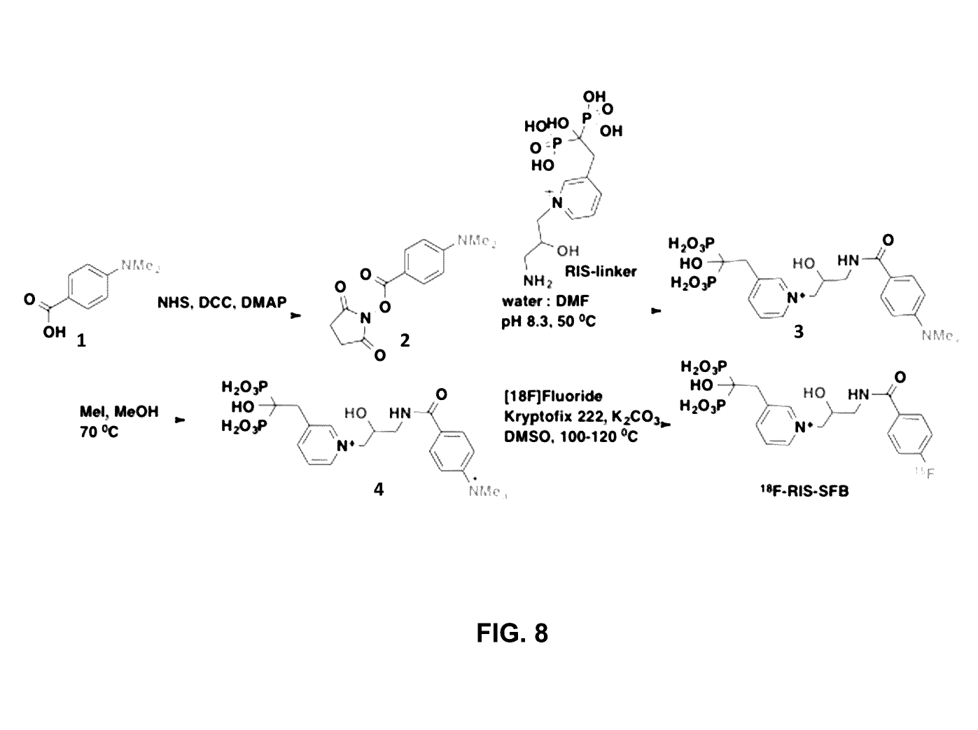

Although the previously described N-succinimidyl ester mediate coupling of the BP and the radiolabel was successful, it can be desirable to incorporate the radiolabel in the last step of production to increase the useful time of the resulting imaging probe. To this end, a synthesis scheme to install radioactive fluorine in the last step was generated. As shown in FIG. 8, 4-dimethylamino benzoic acid (DMABA, FIG. 8, formula 1) was treated with N-hydroxysuccinimide under standard coupling conditions (N,N′-dicyclohexylcarbodiimide (DCC) with catalytic amount of 4′-dimethylaminopyridine (DMAP) in appropriate solvent, e.g., chloroform) to yield succinimidyl benzoate (FIG. 8, formula 2). The activated ester was treated with a BP-linker conjugate (e.g., FIG. 8, RIS-linker) to generate the BP-DMABA conjugate (FIG. 8, formula 3). Methyl iodide in methanol was used quaternize the BP-DMABA conjugate, and the quaternized compound (FIG. 8, formula 4) was then further reacted with the radiolabel to yield the final single modality ¹⁸F-BP-SFB conjugate probe.

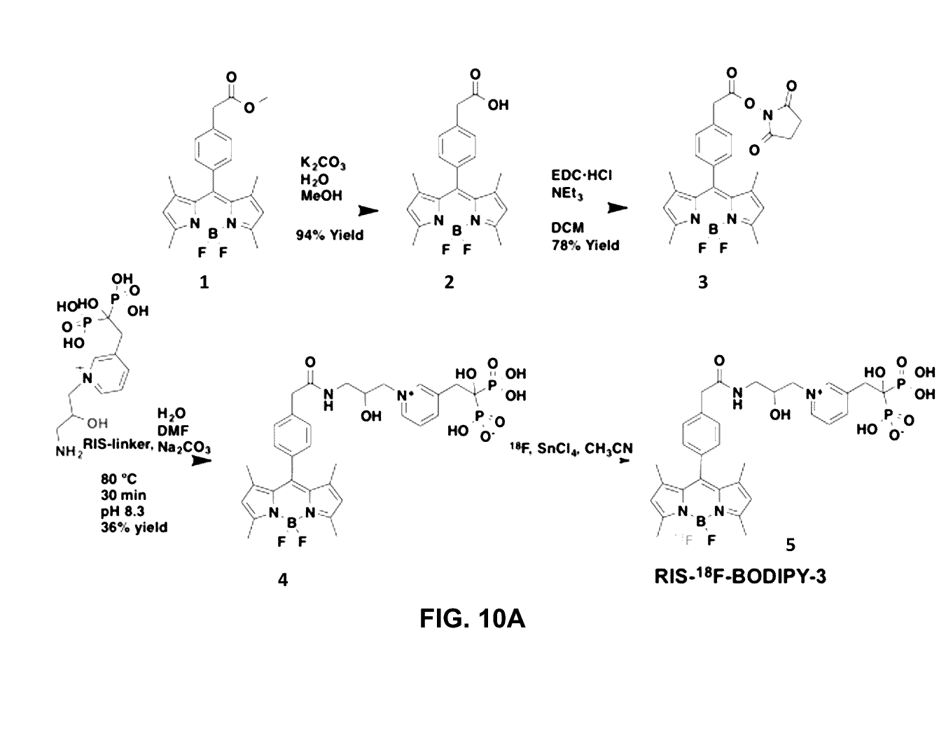

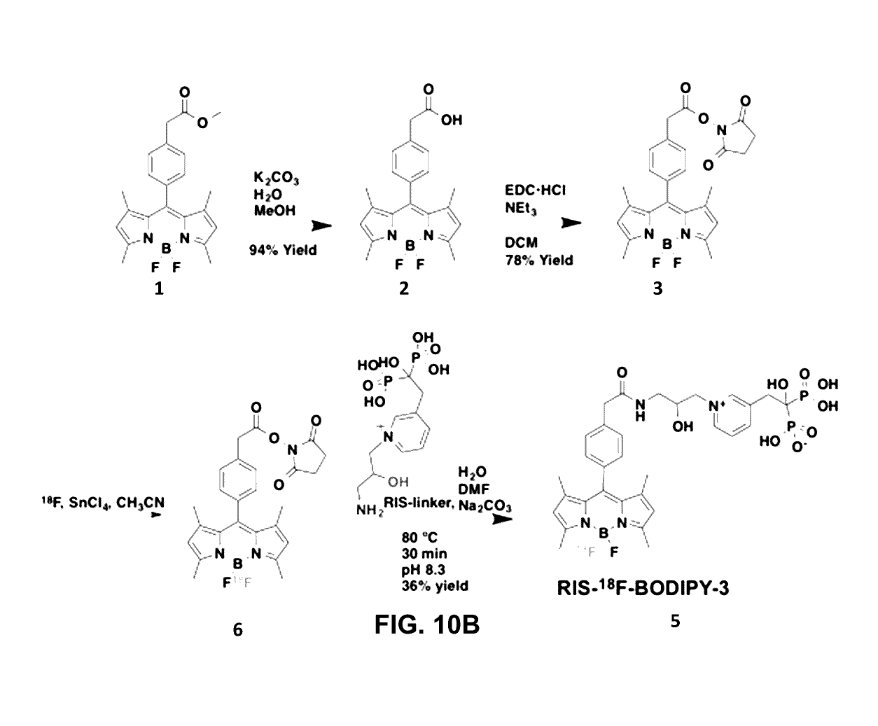

To produce the dual modality BP conjugated probes, a fluoride exchange approach was utilized (Li et al. Rapid aqueous F-18-labeling of a bodipy dye for positron emission tomography/fluorescence dual modality imaging. Chemical Communications. 2011; 47(33):9324-6 and Liu et al. Lewis Acid-Assisted Isotopic F-18-F-19 Exchange in BODIPY Dyes: Facile Generation of Positron Emission Tomography/Fluorescence Dual Modality Agents for Tumor Imaging. Theranostics. 2013; 3(3):181-9). Briefly, the BODIPY methyl (FIG. 10A, formula 1) ester was synthesized similarly according to the published literature (e.g. FIG. 10A). Saponification of FIG. 10A, formula 1 with potassium carbonate smoothly provided the acid (FIG. 10A, formula 2), which was then converted with a good yield to N-succinimidyl ester form (FIG. 10A, formula 3) by standard coupling conditions (1-(3-dimethylaminopropyl)-3-ethylcarbodiimide HCl (EDC.HCl) as coupling reagent with triethylamine (NEt₃) as base, in appropriate solvents, e.g., dichloromethane). The full elaborated dual modality BP conjugated imaging probe (e.g., FIG. 10A, formula 5) was produced via base promoted coupling of the BODIPY succinimidyl ester (FIG. 10A, formula 3) to the BP (e.g. RIS or ZOL)-linker (e.g., FIG. 7, RIS-linker), followed by the treatment of the resulted FIG. 10A, compound 4, under conditions established by Li et al., Chem Comm., 2011, 47: 9324-9326 and Li et al., 2013. Theranostics. 3(3): 181-189, which are incorporated by reference herein as if expressed in their entirety. The radiolabeled BODIPY dual modality probe (FIG. 10B, formula 5) was also successfully synthesized through the conjugation of the BP-linker (FIG. 7, RIS-linker) with radiolabeled (e.g. ¹⁸F) BODIPY compound (FIG. 10B, formula 6).

### Example 2

### PET Imaging with Single Modality BP Conjugated Imaging Probes

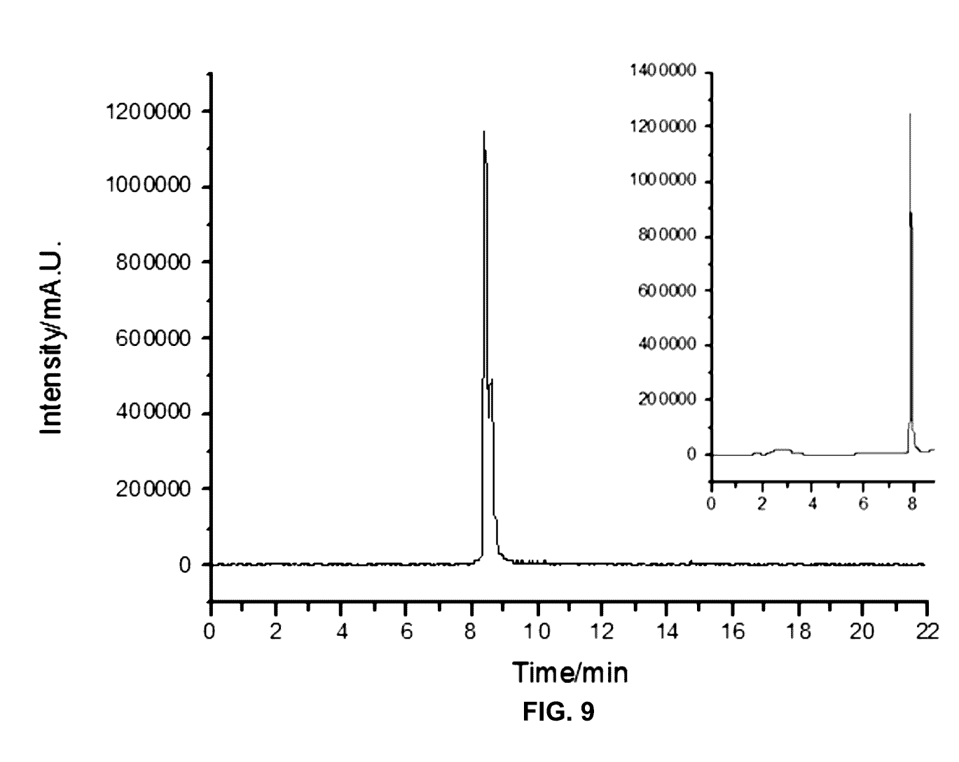

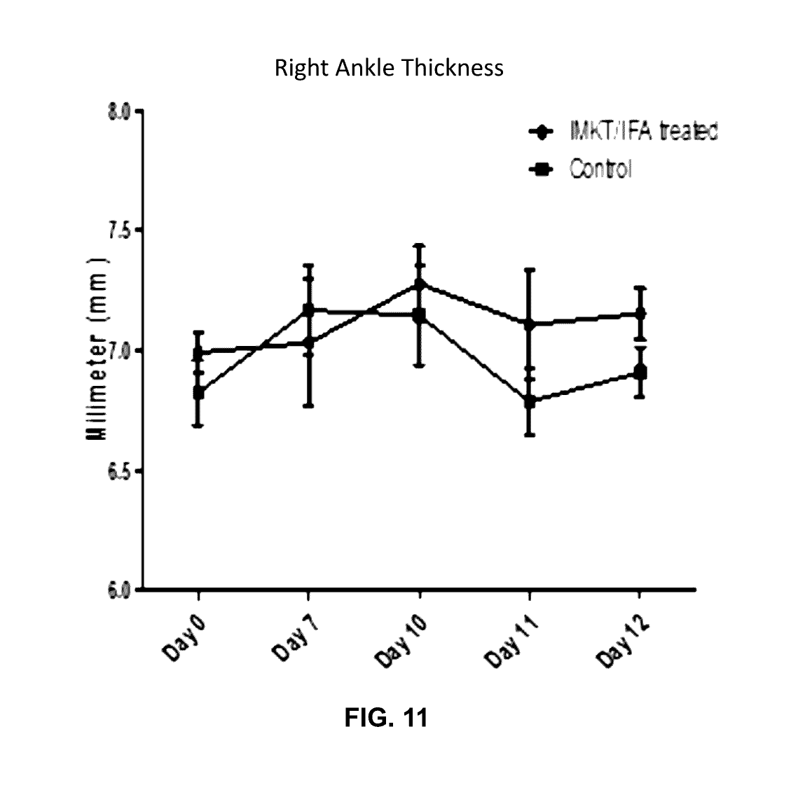

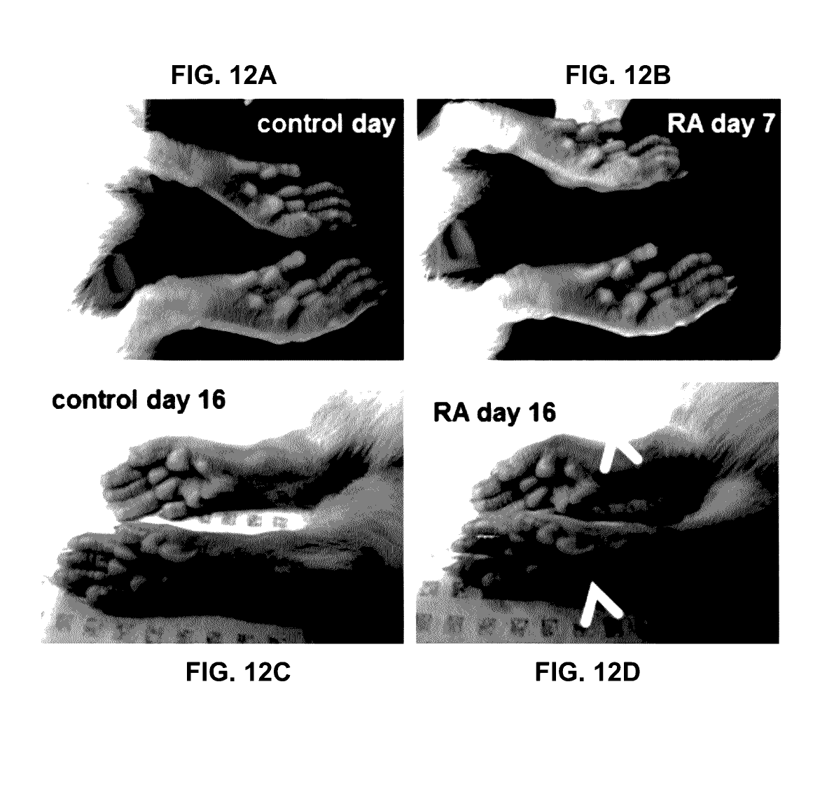

Single modality BP imaging probes with ¹⁸F were generated according to the Schemes described in FIGS. 6A-6B and FIG. 7. FIG. 9 shows a graph that demonstrates that ¹⁸F-RIS-SFB imaging probes were generated with an acceptable yield and purity. ¹⁸F-ZOL-SFB imaging probes were also generated with a similar procedure. A rat (Lewis rats, 6-12 weeks of age) model of arthritis was generated by subcutaneous injection of heat killed Mycobacterium tuberculosis (strain H37Ra) suspended in incomplete Freund's adjuvant (HKMT/IFA). In this model animals displayed clinically visible signs of inflammation in the ankle and paw that became measurable significantly at day 12 post injection (FIG. 11). The swelling was easily identifiable by day 16 post injection, but undetectable in the animals on day 7 (FIGS. 12A-12D).

Rats were given a total of about 500 μCi (about 0.5 nmole) of freshly prepared ¹⁸F-RIS-SFB (single modality BP conjugated imaging probe) by i.v. injection. PET imaging was conducted using a GE eXplore Vista small animal PET system.

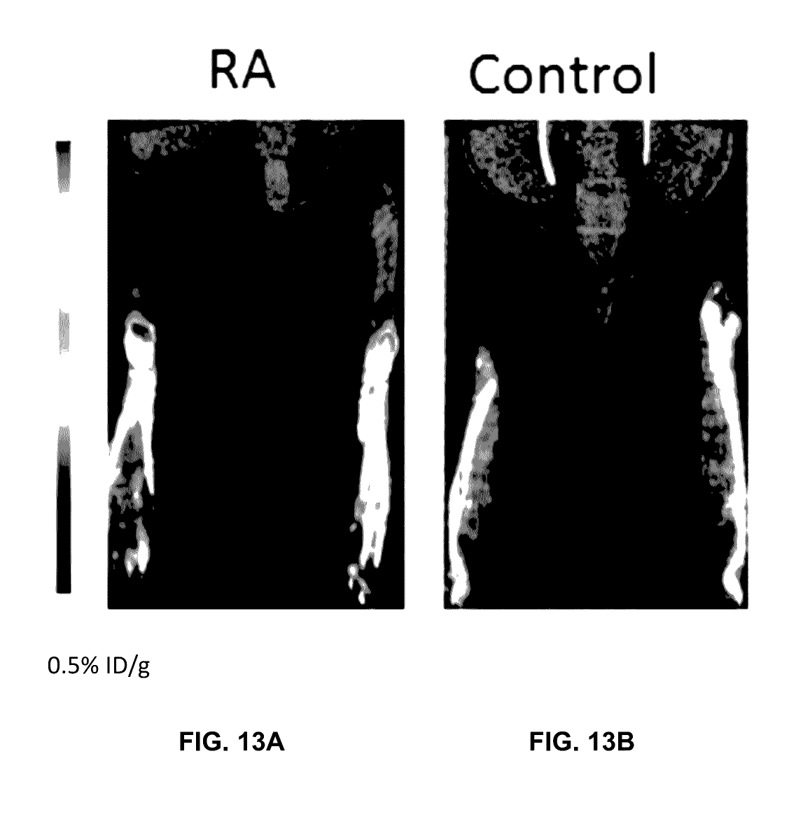

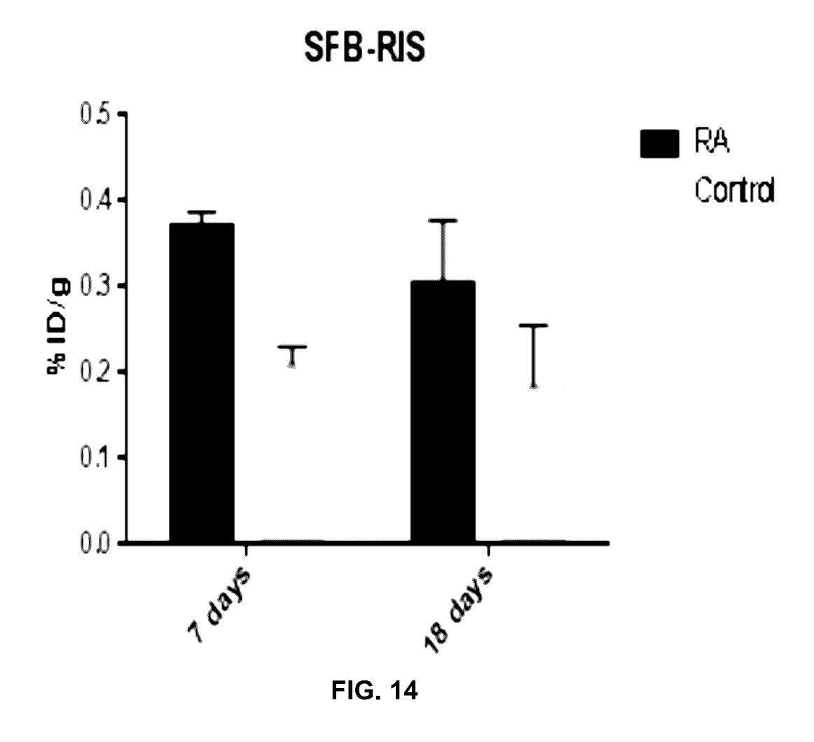

As can be observed from representative PET scan images (FIGS. 13A and 13B) of animals injected with ¹⁸F-RIS-SFB, significant (P=0.0002) differences in the bone uptake in these animals was readily detect at day 7 (FIG. 14).

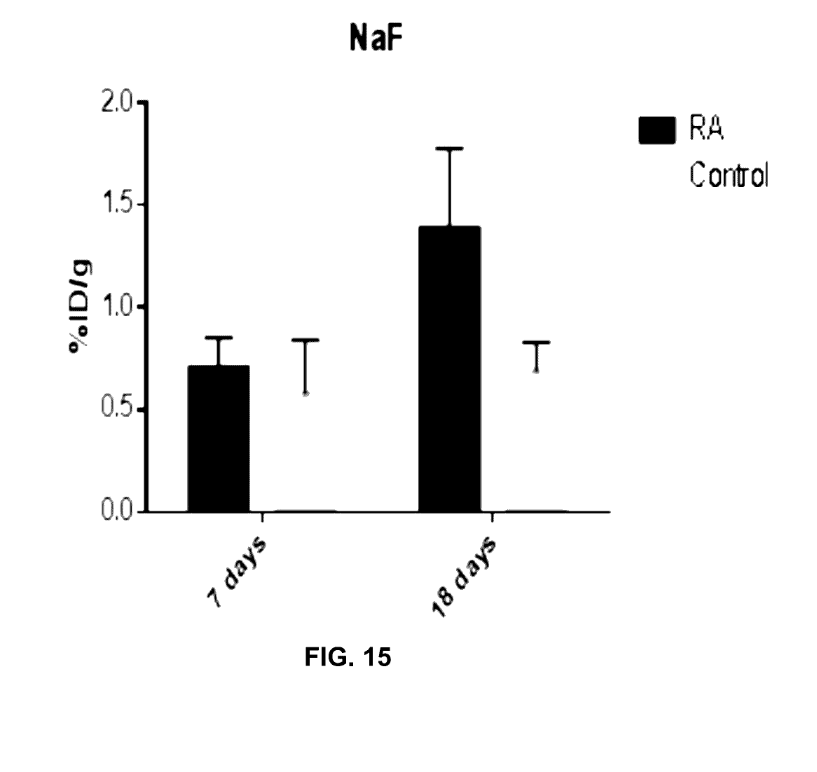

¹⁸F-labeled fluoride has recently been explored as a PET imaging compound for RA. Because fluoride accumulates in bone, Na¹⁸F shows differences in tissue accumulation and metabolic signaling independent of the level of ¹⁸F-deoxyglucose. Interestingly, ^{18F} has not been observed to increase in uptake prior to clinical symptoms of inflammatory RA and thus it has been previously concluded that autoimmune inflammation precedes bone erosive activity. In the model of RA described in the present Example Na¹⁸F fails to detect the earliest signs of bone involvement in RA and only shows an increased accumulation in the bone later (about day 18). See FIG. 15. These results with Na¹⁸F is in sharp contrast to what can be observed using ¹⁸F-RIS-SFB, which allows for imaging bone involvement in RA at an earlier, and in some cases pre-clinical, time point.

### Example 3

### PET Imaging with Dual Modality BP Conjugated Imaging Probes

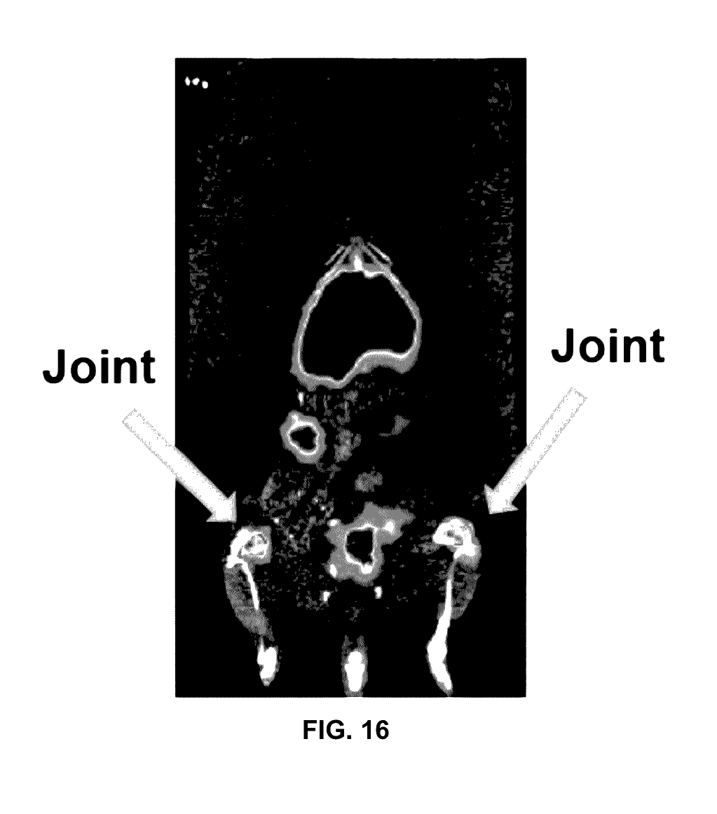

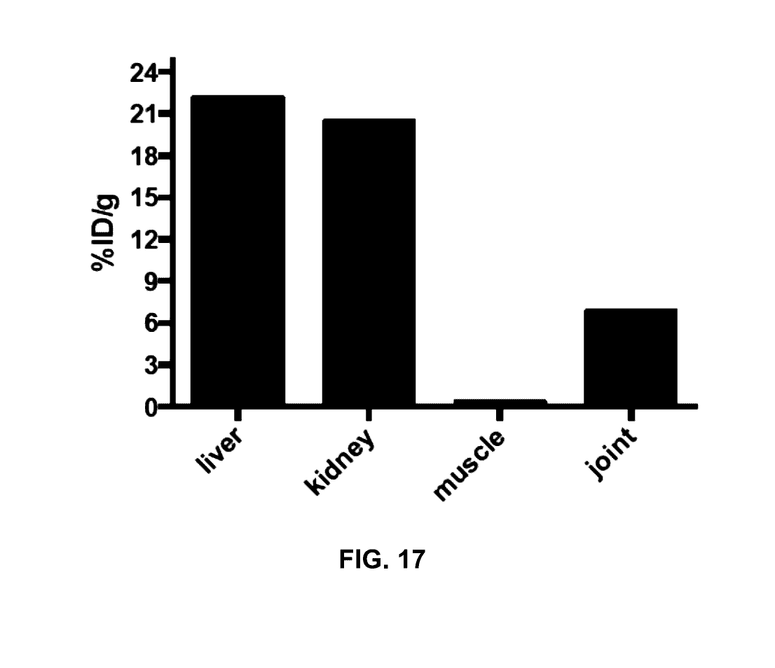

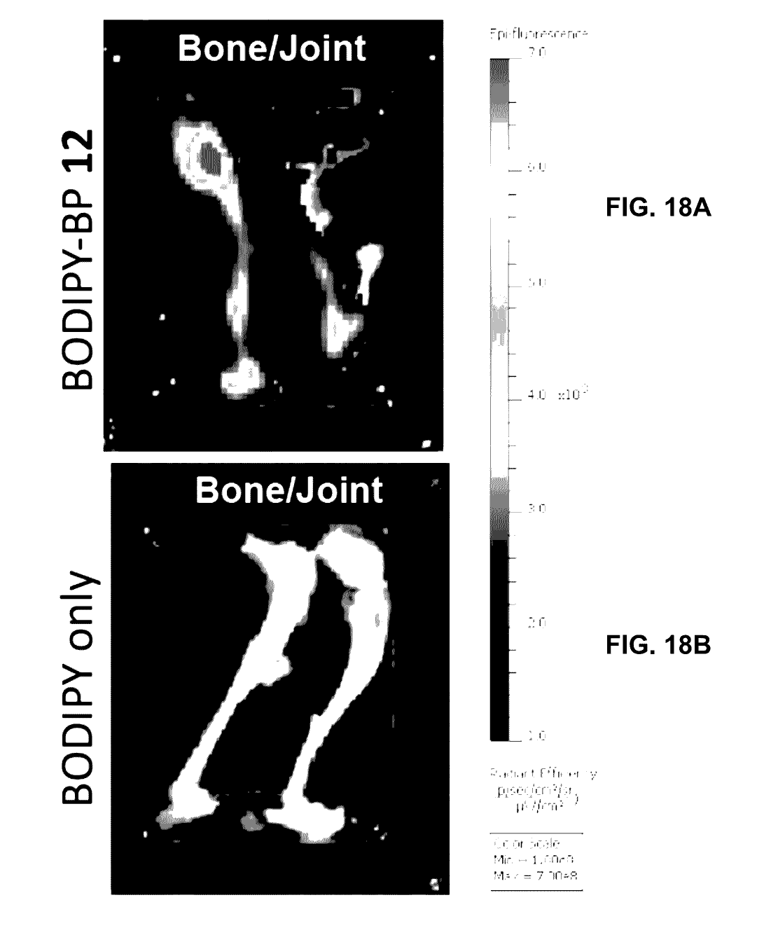

Dual modality BP-conjugated imaging probes were generated as previously described in Example 1. Uptake of the ¹⁸F-BODIPY-RIS probe in a normal (non-RA) mouse was evaluated. Briefly, about 150 μCi of the probe was administered i.v. and about 1 hour post injection the animals underwent a PET scan. As shown in FIGS. 16-17, strong uptake into various tissues was observed by PET scanning. The compound was observed to be distributed to the joints, as well as to the liver, kidney, and some skeletal muscle. This indicated that this is a feasible route to administer BP-conjugated PET agents. These results were confirmed using ex vivo imaging. As demonstrated by the representative images of FIGS. 18A and 18B, only the BP conjugated dual modality probe (FIG. 18A) demonstrated prominent fluorescent signal at the joints as compared to the BODIPY dye itself (FIG. 18B).

### Example 4

### Synthesis of Near Infrared BODIPY BP Conjugated Imaging Probes

There has been increasing interest in development of far-red and NIR emissive fluorescent dyes in bio-imaging in living systems. Fluorescence in the long-wavelength region generates minimal photo-toxicity to biological components, and has deep tissue penetration and minimal background signal from biomolecular auto-fluorescence. BODIPY dyes have received considerable attention as useful imaging probes due to their excellent photo-physical properties and potential for fluorescence-based sensing and bio-imaging applications. In addition, the possibility to add ¹⁸F in a BODIPY scaffold by direct ¹⁹F-¹⁸F exchange as described elsewhere herein, makes them a candidate for PET-BP dual modality imaging probes.

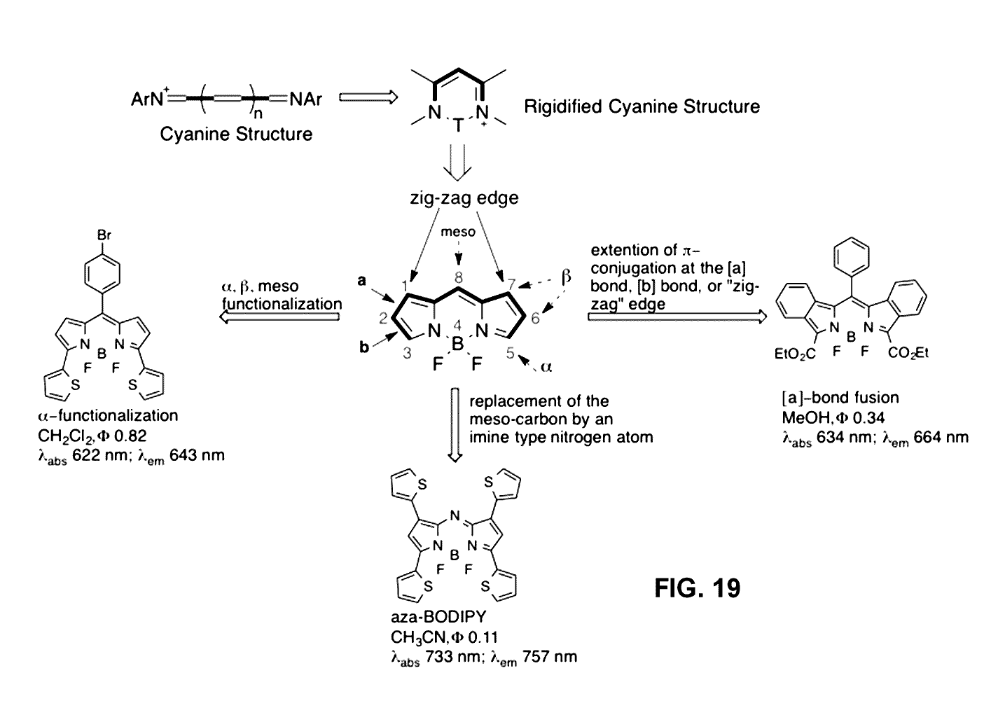

A shown in FIG. 19, the BODIPY structure is an example of a “rigidified” mono-methine cyanine. Its greatly restricted flexibility leads to unusually high fluorescence quantum yields from the dipyrromethene-boron framework. The π-electrons delocalize along the organic backbone and can be further extended by substitution or fusion of aromatic units to one or both pyrrole fragments. Such an extended delocalization pathway can be used to obtain dyes with fluorescence in the far-red or NIR spectral region. Strategies to extend the π-conjugation can be grouped into three categories and one example is provided for each strategy in FIG. 19. The first category of strategies employs functionalization at the α-(position 3, 5), β-(position 1, 2, 6, 7) and meso-(position 8) sites of the BODIPY core to extend π-conjugation and to generate a “push-pull” structure. The category of strategies utilizes employment of π-extended pyrrole units or fusion of aromatic units to extend the π-conjugation at the [a] bond, [b] bond and the “zig-zag” edge of the BODIPY. The third category of strategies employs replacement of the meso-carbon by an imine nitrogen atom. It is noteworthy that these strategies can also be combined in the design of one molecule. Thus, the introduction of molecular rigidity and some electron-donating groups, such as dialkylamino or alkoxy could result in even more pronounced spectral changes.

Another factor to consider in the design of the NIR BODIPY PET-Fluor BP probes is water-solubility. Most of the BODIPY dyes are soluble in organic solvents, but not in water. The BP-linker is very hydrophilic and an aqueous DMF/DMSO/MeOH solution is usually needed in the conjugation reaction of BP-linker and BODIPY dyes. Although the BP-¹⁸F-BODIPY probe synthesis was successful despite that fact that BODIPY did not completely dissolve in the aqueous DMSO solution, a certain degree of water solubility can be desirable in the design of the NIR BODIPY dye. This can be especially true with a more hydrophobic scaffold due to the extended π-conjugation system. In some instances, various hydrophilic groups can be introduced, such as quaternary ammonium, sulfonate, phosphonate or oligo ethyleneglycol moieties, into the BODIPY core. The solubility of these dyes in aqueous solution can be greatly improved while maintaining their high fluorescence quantum yields.

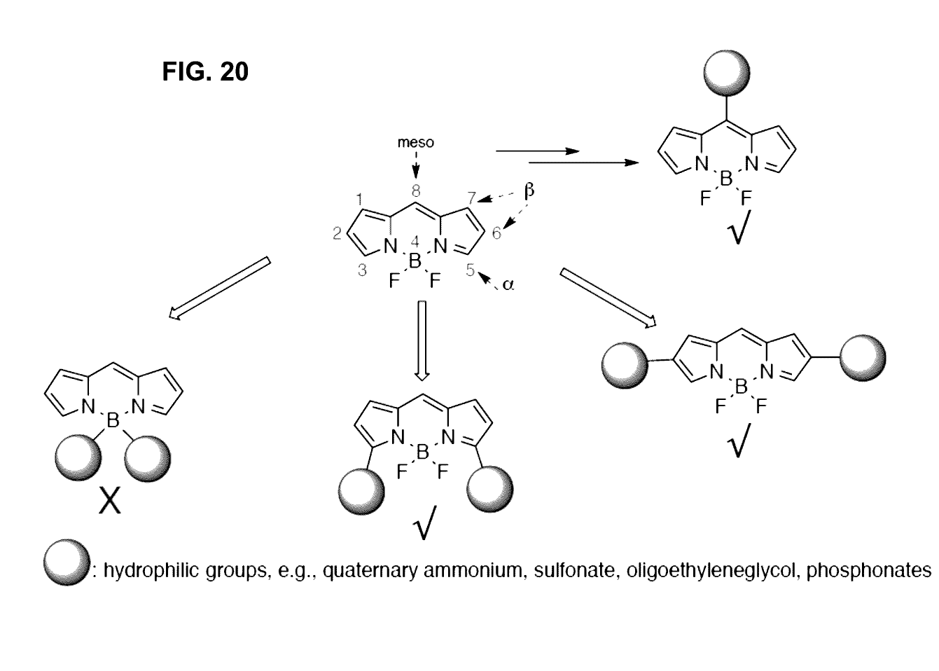

The hydrophilic groups can be added to any of the positions (1-8) of the BODIPY structure (FIG. 20). However, according to the commonly used approaches to synthesize BODIPY dyes, positions 2-6 are preferred to introduce the hydrophilic groups. Introduction to the meso-position was also reported, but the hydrophilic groups were introduced after the BODIPY core structures were constructed. As a B—F bond is used for the PET-Fluor BP dual-modality probes, introduction of a hydrophilic group at position 4 (boron) is not appropriate in these probes.

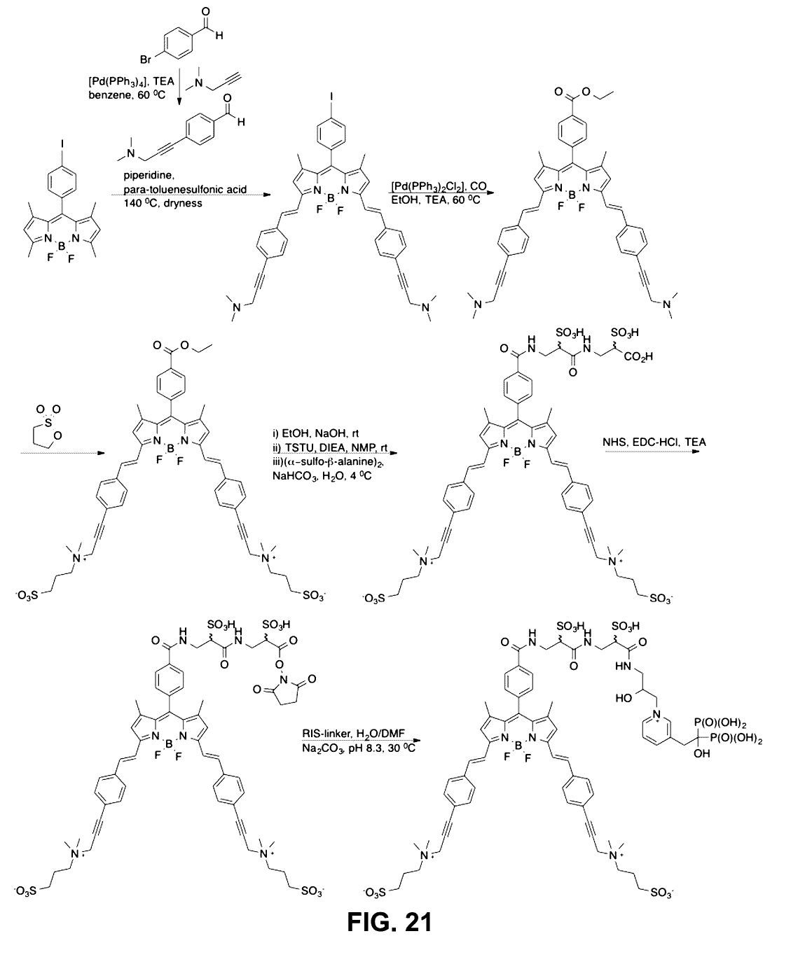

FIG. 21 summarizes the design considerations discussed above and demonstrates the overall synthetic route of NIR-BODIPY-F-BP using cold F, however, the same approach applies to hot F (¹⁸F) with the additional step of generating the radioactive ¹⁸F via the Lewis Acid-assisted isotopic ¹⁸F-¹⁹F exchange described elsewhere herein.

Ratios, concentrations, amounts, and other numerical data herein may be expressed in a range format. It is to be understood that such a range format is used for convenience and brevity, and should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1% to about 5%, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, the term “about” can include traditional rounding according to significant figure of the numerical value. In addition, the phrase “about ‘x’ to ‘y’” includes “about ‘x’ to about ‘y’”.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

We claim:

1. An imaging probe comprising:
a bisphosphonate; and
a positron emission tomography (PET) radionuclide, wherein the PET radionuclide is conjugated to the bisphosphonate.

2. The imaging probe of claim 1, wherein the PET radionuclide is directly conjugated to the bisphosphonate via N-succinimidyl-4-[¹⁸F]fluorobenzoate and an epoxy-containing linker.

3. The imaging probe of claim 1, wherein the PET radionuclide conjugate is synthesized from N-succinimidyl-4-[¹⁸F]fluorobenzoate bound to a bisphosphonate through an epoxy-containing linker.

4. The imaging probe of claim 1, wherein the PET radionuclide is selected from the group consisting of: ¹⁸F, ¹¹C, ⁶⁰Cu, ⁶¹Cu, ⁶⁴Cu, ⁸⁶Y, ¹²⁴I, and ⁸⁹Zr.

5. The imaging probe of claim 1, wherein the bisphosphonate is a nitrogen containing bisphosphonate.

6. The imaging probe of claim 1, wherein the bisphosphonate is a non-nitrogen containing bisphosphonate.

7. The imaging probe of claim 1, wherein the bisphosphonate is selected from the group consisting of risedronate, zoledronate, minodronate, pamidronate, neridronate, olpadronate, alendronate, ibandronate and analogues thereof.

8. The imaging probe of claim 1, wherein an alpha-hydroxyl of the bisphosphonate is substituted with H.

9. The imaging probe of claim 8, wherein the bisphosphonate is (2-(pyridin-4-yl)ethane-1,1-diyl)bis(phosphonic acid) or a salt thereof.

10. The imaging probe of claim 1, wherein the bisphosphonate is (1-hydroxy-2-(pyridin-4-yl)ethane-1,1-diyl)bis(phosphonic acid) or a salt thereof.

11. The imaging probe of claim 1, further comprising a fluorescent molecule, wherein the fluorescent molecule is conjugated to the bisphosphonate via an epoxy-containing linker and wherein the PET radionuclide is directly conjugated to the fluorescent molecule.

12. The imaging probe of claim 11, wherein the fluorescent molecule is a boron-dipyrromethene (BPDIPY) dye.

13. The imaging probe of claim 11, wherein the bisphosphonate is a nitrogen containing bisphosphonate.

14. The imaging probe of claim 11, wherein the bisphosphonate is a non nitrogen containing bisphosphonate.

15. The imaging probe of claim 11, wherein the bisphosphonate is selected from the group consisting of risedronate, zoledronate, minodronate, pamidronate, neridronate, olpadronate, alendronate, ibandronate and analogues thereof.

16. The imaging probe of claim 11, wherein the alpha-hydroxyl of the bisphosphonate is substituted with H.

17. The imaging probe of claim 16, wherein the bisphosphonate is (2-(pyridin-4-yl)ethane-1,1-diyl)bis(phosphonic acid or a salt thereof.

18. The imaging probe of claim 11, wherein the bisphosphonate is (1-hydroxy-2-(pyridin-4-yl)ethane-1,1-diyl)bis(phosphonic acid) or a salt thereof.

19. A method comprising:
administering an imaging probe to a subject in need thereof, wherein the imaging probe comprises:
a bisphosphonate; and
a positron emission tomography (PET) radionuclide, wherein the PET radionuclide is conjugated to the bisphosphonate; and
obtaining an image of at least a portion of the subject using PET scanning.

20. The method of claim 19, wherein the PET radionuclide is directly conjugated to the bisphosphonate via N-succinimidyl-4-[¹⁸F]fluorobenzoate and an epoxy-containing linker.

21. The method of claim 19, wherein the imaging probe further comprises a fluorescent molecule, wherein the fluorescent molecule is conjugated to the bisphosphonate via an epoxy-containing linker and wherein the PET radionuclide is directly conjugated to the fluorescent molecule.

22. The method of claim 19, wherein the PET radionuclide is selected from the group consisting of: ¹⁸F, ¹¹C, ⁶⁰Cu, ⁶¹Cu, ⁶⁴Cu, ⁸⁶Y, ¹²⁴I, and ⁸⁹Zr.

23. The method of claim 19, wherein the subject in need thereof has, is suspected of having, or is otherwise predisposed to having a bone related disease, wherein the bone related disease is selected from the group consisting of: multiple myeloma, bone metastasis, Paget's disease, steroid induced osteoporosis, osteosarcoma osteoporosis, osteopenia, heterotopic ossification, osteoarthritis, rheumatoid arthritis, a disorder characterized by high bone turnover, and any combination thereof.
